(19) United States
(12) Patent Application Publication
(10) Pub. No.: US 2025/0298034 A1
(43) Pub. Date: Sep. 25, 2025

(54) METHODS OF TREATING IMMUNOTHERAPY-ASSOCIATED ADVERSE EFFECTS

(71) Applicant: NORTHWESTERN UNIVERSITY, Evanston, IL (US)

(72) Inventor: Jennifer D. Wu, Wilmette, IL (US)

(21) Appl. No.: 18/862,873

(22) PCT Filed: May 5, 2023
(86) PCT No.: PCT/US23/66680

§ 371 Date: Nov. 4, 2024

Related U.S. Application Data
(60) Provisional application No. 63/338,651, filed on May 5, 2022.

(51) Int. Cl. G01N 33/68; A61K 31/573

(52) U.S. Cl. CPC G01N 33/6872; A61K 31/573; G01N 33/6869; G01N 2333/54; G01N 2333/70539; G01N 2800/52

(57) ABSTRACT

Described herein are methods of treating an immunotherapy-associated adverse event in a subject in need thereof comprising detecting a level of NKG2D receptor ligand polypeptide (and optionally IL-18) in a sample form the subject; and (b) administering either (1) a corticosteroid or (2) a TNFα inhibitor or an integrin inhibitor to the subject.

### CROSS-REFERENCE TO RELATED APPLICATIONS

This application claims priority to U.S. Provisional Application No. 63/338,651, filed May 5, 2022, which is incorporated herein by reference in its entirety.

### STATEMENT OF GOVERNMENT SUPPORT

This invention was made with government support under R01CA208246-05 awarded by the National Institutes of Health (NIH). The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2021-228_Seqlisting.xml”, which was created on May 3, 2023 and 86,243 bytes in size. The subject matter of the Sequence Listing is incorporated herein in its entirety by reference.

### BACKGROUND

Immunotherapy with immune checkpoint inhibitors (ICI) targeting cytotoxic T lymphocyte-associated antigen 4 (CTLA4) and programmed cell death protein 1 or its ligand (PD1)/PDL1 has transformed the treatment of an expanding list of malignancies with overall improvement in progression-free and overall survival for many cancer patients. Despite important clinical benefits, immune checkpoint inhibition has been associated with a unique spectrum of toxicities termed immune-related adverse events (irAEs) in a broad spectrum of organs¹. The development of severe gastrointestinal inflammation, primarily in the form of colitis and diarrhea, is one of the most significant irAEs in patients receiving ICI single agent of anti-PD1/PDL1 or anti-CTLA4 or in combination, with the combination therapy inducing more severe and more frequent toxicity²⁻⁶. While low severity irAE, such as skin rash and hypothyroidism, has been associated with improved cancer outcome in a subset of patients, recent reports suggest that long term corticosteroid management could adversely impact clinical outcome of ICI therapy⁷⁻¹⁰. The understanding of underlying mechanisms associated with ICI-associated colitis (ICI-colitis) is limited. Current mainstay approaches to manage the ICI-colitis are adapted from the treatment for inflammatory bowel disease (IBD) or ulcerative colitis (UC)^{7,11-13}. Based upon empiric clinical experiences, corticosteroids are the recommended initial treatment for ICI-colitis with anti-TNFα (infliximab) or anti-α47 (vedolizumab) being the 2^{nd} line for corticosteroids refractory patients¹⁴⁻¹⁷. With the multiple steps of immune suppressant management, ICI therapy often is significantly delayed if resumption is achievable, or therapy is discontinued⁷. Further, prolonged use of these immune suppressants could increase the risk of infection in patients^{15,18}. Thus, there remains a need in the art to shorten the duration of immune suppression for ICI-colitis management, and improve ICI therapy for cancer patients.

### SUMMARY

In one aspect, described herein is a method of treating an immunotherapy-associated adverse event in a subject in need thereof comprising (a) detecting an elevated level of NKG2D receptor ligand polypeptide in a sample from the subject; and (b) administering a corticosteroid to the subject. In some embodiments, the NKG2D receptor ligand polypeptide is a soluble Major Histocompatibility Complex class I chain-related (sMIC) polypeptide or an UL binding protein (ULBP). In some embodiments, the ULBP is ULBP-1, ULBP-2, UL-BP-3, ULBP-4, ULBP-5 or ULBP-6. In some embodiments, the NKG2D receptor ligand polypeptide is a soluble Major Histocompatibility Complex class I chain-related (sMIC) polypeptide. In some embodiments, the elevated level of sMIC in the sample comprises an amount that is ≥10% increase compared to baseline. In some embodiments, the method further comprises detecting an elevated level of IL-18 in the sample. In some embodiments, the elevated level of IL-18 in the sample comprises an amount that is ≥90 pg/mL.

In another aspect, described herein is a method of treating an immunotherapy-associated adverse event in a subject in need thereof comprising (a) detecting a decreased level of NKG2D receptor ligand polypeptide in a sample from the subject; and (b) administering a TNFα inhibitor or an integrin inhibitor to the subject. In some embodiments, the NKG2D receptor ligand polypeptide is a soluble Major Histocompatibility Complex class I chain-related (sMIC) polypeptide or an UL binding protein (ULBP). In some embodiments, the ULBP is ULBP-1, ULBP-2, UL-BP-3, ULBP-4, ULBP-5 or ULBP-6. In some embodiments, the NKG2D receptor ligand polypeptide is a soluble Major Histocompatibility Complex class I chain-related (sMIC) polypeptide. In some embodiments, the decreased level of sMIC in the sample comprises an amount that is ≥10% lower compared to baseline. In some embodiments, the method further comprises detecting a decreased level of IL-18 in the sample.

### BRIEF DESCRIPTION OF THE FIGURES

FIG. 1A is immunohistochemistry (IHC) staining with 1.0 ug/ml of anti-MIC/sMIC antibody (sc-271535, Santa Cruz Biotechnology) of the formalin-fixed paraffin embedded (FFPE) colon biopsy sections from cancer patients who received immune checkpoint inhibitor (ICI) therapy. The brown color represents the positive staining of sMIC. NHC, no histological evidence of colitis; ICI-colitis-sMICHi, high score of MIC/sMIC staining as quantified in (b) of ICI-colitis colon. ICI-colitis-sMICLo, low score of MIC/sMIC staining as quantified in (b) of ICI-colitis colon. Standard IHC staining of FFPE protocol was used. FIG. 1B is a graph showing semi-quantitative MIC IHC staining scores on the selected ROIs for DSP analysis from both anti-CTLA4+anti-PD1 combination therapy induced colitis (Combo) and anti-PD1 monotherapy induced colitis (aPD1).

FIG. 2 is a graph showing serum sMIC (measured by ELISA) in patients with whom colon MIC was assessed by IHC as high (Hi) or low (Lo). Serum levels of sMIC (SMIC-A and sMIC-B) in patients of NHC, colon sMICHi by IHC and colon sMICLo by IHC as detected by IHC. The value presented is the combined value of sMIC-A and sMIC-B as measured with the human MIA DuoSet and human MIC-B DuoSet ELISA from R&D systems respectively. Data show that patients with high colon sMIC IHC score also have high levels of serum sMIC.

FIGS. 3A-3E Quantification of different cell type or subtype from mIHC CD8 toxic panel were performed on entire FFPE section of each individual colon sample. Cell type frequency was measured as the proportion of the interested cell type from total cells on the same image (DAPI positive, segmented with inform software). Average of cell type frequencies from the same colon biopsy sample was computed and presented as a data point in dot plot. p value of t-test between different group are shown. Combo (dark grey dot, left column of data on plot): patient received anti-CTLA4 and anti-PD1 combination therapy. aPD1 (light grey dot, right column of data on plot): patient received anti-PD1 therapy only FIG. 3A is a dot plot showing the percentage of CD8 T cells. FIG. 3B is a dot plot showing the percentage of granzyme B (GzmB)-positive cells. FIG. 3C is a dot plot showing the percentage of granzyme B-positive (GzmB+) CD8 T cells. FIG. 3D is a dot plot showing the percentage of CD103+CD8T cells. FIG. 3E is a dot plot showing the percentage of CD103+ GzmB+CD8T cells.

FIGS. 4A-4E show the quantification of antigen presenting CD68+ and CD11c+ cells (APCs) from entire FFPE section of each individual colon sample that were performed with mIHC and in vitro assay presenting that sMIC induction of PD-L1 expression on APCs/macrophages. Cell type frequency was measured as the proportion of the interested cell type from total cells (DAPI positive). Average of cell type frequencies from the same colon biopsy sample was computed and presented as a data point in dot plot. p value of t-test between different group are shown. Colitis biopsy samples from patients treated with ICI combination therapy are shown in red and samples from ICI monotherapy were shown in green. Combo (dark grey dot, left column of data on plot): patient received anti-CTLA4 and anti-PD1 combination therapy. aPD1 (light grey dot, right column of data on plot): patient received anti-PD1 therapy only. FIG. 4A is a dot plot showing the percentage of CB68⁺ and CF11c⁺ cells (APCs) as assessed by mIHC. FIG. 4B is a dot plot showing the percentage of PDL1+ APCs as assessed by mIHC. FIG. 4C is a dot plot showing the frequency of granzyme B+ CD8+ T cells who are in direct contact with APCs among total granzyme B+CD8+ T cells. FIG. 4D is a graph showing the significant correlation of MIC IHC staining scores with the frequency of PD-L1+ antigen presenting cell in ICI colitis induced both by combination therapy and by monotherapy. FIG. 4E is a histogram of flow cytometry showing PDL1 expression on human monocyte-derived M1 macrophages.

FIG. 5 is a summary of sMIC level in the colon, enriched inflammatory pathways in ICI-colitis colon, and patients' responses to corticosteroid and 2^{nd} line of therapy.

FIG. 6 provides graphs showing serum levels of IL-18 in ICI-colitis patients with colon MICHi and different responses to corticosteroid treatment. Data show that, among colon MICHi ICI-patients, only those with high levels of serum IL-18 were responsive to corticosteroid treatment.

FIG. 7 are IHC image showing increased IL-18-expression in the colon ICI-colitis patients who had high serum/colon levels of sMIC and IL-18 (sMIC^{Hi}/serum IL-18^{Hi}). Colon tissues were stained by IHC with an anti-human IL-18 antibody.

FIG. 8 provides a summary of the data provided in the Examples and proposed model of pathway-guided personalized treatment for ICI-colitis patients.

FIG. 9 provides a list of reagents being used for multicolor immunohistochemistry (mIHC) assay.

FIG. 10 depicts serum levels of IL-18 in cancer patients who received immune checkpoint inhibitor (ICI)-therapy. The level of IL-18 was measured using human cytokine 48-Plex Discovery Assay by Eve Technologies, Alberta, Canada. Each dot represents one patient. Statistical analysis was performed using unpaired two-tailed t-test with Welch's correction. P<0.05 is considered statistically significant.

FIG. 11 shows a receiver operating characteristic (ROC) curve that illustrates the predictive ability of serum IL-18 in sMICHi ICI-colitis patients' response to corticosteroid treatment, with p=0.0002 and area under curve (AUC) of 0.986.

### DETAILED DESCRIPTION

The present application is based, in part, on the discovery that ICI-associated colitis patients exhibiting high levels of serum or colonic Major Histocompatibility Complex class I chain-related (sMIC) polypeptide (and optionally high CD8 granzyme activity (observed by the patient having elevated levels of IL-18), have a positive response to corticosteroid treatment. In contrast, patients with low levels of sMIC polypeptide presented shared molecular and cellular features with IBD/UC and responded poorly to corticosteroids yet well to anti-TNFα therapy. These findings provided scientific parameters for developing a biomarker platform to select patients for more effective management of ICI-colitis, thereby improving the outcome of immunotherapy.

### NKG2D Receptor Ligands

Natural killer (NK) cells exhibit on their surfaces the NKG2D receptor, a prominent, homodimeric, surface immunoreceptor responsible for recognizing a target cell and activating the innate defense against the pathologic cell (Lanier, L L, 1998. NK cell receptors. Ann. Rev. Immunol. 16:359-393; Houchins J P et al. 1991, Exp. Med. 173:1017-1020; Bauer, S et al., 1999, Science 285:727-730). The human NKG2D molecule possesses a C-type lectin-like extracellular domain that binds to its cognate ligands. Examples of such ligands include, but are not limited to, MIC-A, MIC-B, heat shock proteins, UL16 binding proteins (e.g., ULBPs 1-6).

Non-pathologic expression of MIC-A and MIC-B is restricted to intestinal epithelium, keratinocytes, endothelial cells and monocytes, but aberrant surface expression of these MIC proteins occurs in response to many types of cellular stress such as proliferation, oxidation and heat shock and marks the cell as pathologic (Groh et al. 1996, PNAS 93:12445-12450; Groh et al. 1998, Science 279:1737-1740; Zwirner et al. 1999, Human Immunol. 60:323-330). Major Histocompatibility Complex class I chain-related (MIC) polypeptides are surface transmembrane proteins. MIC polypeptides include, but are not limited to the human MIC-A (e.g. NCBI Ref Seqs NP_000238 (SEQ ID NO:1) and 001170990) and human MIC-B (e.g. NCBI Ref Seq: NP_005922 (SEQ ID NO: 2). In some embodiments, the MIC polypeptide comprises MIC-A. In some embodiments, the MIC polypeptide can comprise MIC-B. In some embodiments, the MIC polypeptide comprises the following amino acid sequence:

(SEQ ID NO: 3)
EPHSLRYNLTVLSWDGSVQSGFLAEVHLDGQPFLRYDRQKCRAKPQGQW
AEDVLGNKTWDRETRDLTGNGKDLRMTLAHIKDQKEGLHSLQEIRVCEI
HEDNSTRSSQHFYYDGELFLSQNVETEEWTVPQSSRAQTLAMNVRNFLK
EDAMKTKTHYHAMHADCLQELRRYLESSVVLRRRVPPMVNVTRSEALEG
NITVTCGASSFYPRNITLTWRQDGVSLSHDTQQWGDVLPDGNGTYQTWV
ATRICQGEEQRFTCYMEHSGNHSTHPVPS.

In some embodiments, the MIC polypeptide comprises a MIC-A allele set forth in one of SEQ ID NOs: 4-57. In some embodiments, the MIC polypeptide comprises a MIC-B allele set forth in one of SEQ ID NOs: 58-90.

UL16-binding proteins (ULBPs) are a novel family of MHC class I-related molecules (MICs) that were identified based on their ability to bind to the human cytomegalovirus (HCMV) glycoprotein UL16. UL16 also binds to a member of another family of MHC class I-like molecules, MIC-B. The ULBPs and MICs are ligands for NKG2D/DAP10, an activating receptor expressed by natural killer (NK) cells and other immune effector cells, and this interaction can be blocked by UL16. Engagement of NKG2D/DAP10 by ULBPs or MICs expressed on a target cell can overcome an inhibitory signal generated by NK-cell recognition of MHC class I molecules and trigger NK cytotoxicity. ULBPs elicit their effects on NK cells by activating the janus kinase 2, signal transducer and activator of transcription 5, extracellular-signal-regulated kinase mitogen-activated protein kinase and Akt/protein kinase B signal transduction pathways. Although ULBPs alone activate multiple signaling pathways and induce modest cytokine production, ULBPs synergize strongly with interleukin-12 for production of interferon-gamma by NK cells. Exemplary ULBP polypeptides include ULBP-1, ULBP-2, ULBP-3, ULBP-4, ULBP-5 and UL-BP6 described in U.S. Pat. Nos. 6,458,350 and 6,774,224, the disclosures of which are incorporated herein by reference in their entireties. The amino acid sequences of ULBP1-6 are set forth in SEQ ID NOs: 91-96).

### Immunotherapy

As used herein, the term “immunotherapy” refers to a treatment designed to enhance the function of the immune system of a subject or to use transfer of immune cells or of immune molecules (e.g., cytokines) to stop or slow the growth of cancer cells, stop the metastasis of cancer cells, and/or target the cancer cells for cell death in the subject. Exemplary immunotherapies include a monoclonal antibody, a non-specific immunotherapy, an oncolytic virus therapy, adoptive T-cell therapy (e.g., adoptive CD4⁺ or CD8⁺ effector T cell therapy), adoptive natural killer (NK) cell therapy, adoptive NK T cell therapy, CAR T cell therapy and cancer (e.g., tumor) vaccines.

In some embodiments, the immunotherapy comprises an immune checkpoint inhibitor. In some embodiments, the checkpoint inhibitor is a small molecule, an inhibitory nucleic acid, an inhibitory polypeptide, antibody or antigen-binding domain thereof, or antibody reagent. In some embodiments, the immune checkpoint inhibitor is an antibody or antigen-binding domain thereof, or antibody reagent binds an immune checkpoint polypeptide and inhibits its activity. Common immune checkpoints that are targeted for therapeutics include, but are not limited to PD-L1, PD-L2, PD-1, CTLA-4, TIM-3, LAG-3, VISTA, and TIGIT. In some embodiments, the immune checkpoint inhibitor is an antibody or antigen-binding domain thereof, or antibody reagent that binds a PD-1, PD-L1, or PD-L2 polypeptide and inhibits its activity.

Inhibitors of known immune checkpoint regulators (e.g., PD-L1, PD-L2, PD-1, CTLA-4, TIM-3, LAG-3, VISTA, or TIGIT) are known in the art. Non-limiting examples of checkpoint inhibitors (with checkpoint targets and manufacturers noted in parentheses) can include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); avelumab (PD-L1; Merck); durvalumab (PD-L1; Medimmune); and TSR-022 (TIM3; Tesaro).

In some embodiments, the immune checkpoint inhibitor inhibits PD-1. Exemplary PD-1 inhibitors include, but are not limited to pembrolizumab (KEYTRUDA®), nivolumab, AUNP-12, and pidilizumab. In another embodiment, the checkpoint inhibitor inhibits PD-L1. Exemplary PD-L1 inhibitors include, but are not limited to atezolizumab, MPDL3280A, avelumab, and durvalumab.

Programmed death-ligand 1 (PD-L1; also known as cluster of differentiation 274 (CD274) or B7 homolog 1 (B7-H1)) is a transmembrane protein that functions to suppress the immune system in particular events such as pregnancy, tissue allografts, autoimmune disease, and hepatitis. Binding of PD-L1 to its receptor programmed death-1 (PD-1) transmits an inhibitory signal that reduces the proliferation of T cells and can induce apoptosis. Aberrant PD-L1 and/or PD-1 expression has been shown to promote cancer cell evasion in various tumors. PD-L1/PD-I blockade can be accomplished by a variety of mechanisms including antibodies that bind PD-I or its ligand, PD-L1. Examples of PD-I and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699; which are incorporated by reference herein in their entireties. In certain embodiments, the PD-1 inhibitors include anti-PD-L1 antibodies. PD-1 inhibitors include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade.

In some embodiments, the immunotherapy comprises a non-specific immunotherapy. Two common non-specific immunotherapies include, e.g., interferons and interleukins. Interferons (such as Roferon-A [2α], Intron A [2β], Alferon [2α]) boost the immune system to target cancer cells for programmed cell death, and/or slow the growth of cancer cells. Interleukins (such as interleukin-2, IL-2, or aldesleukin (Proleukin)) boost the immune system to produce cells that target cancer cells for programmed cell death. Interleukins are used to treat, e.g., kidney cancer and skin cancer, including melanoma.

In some embodiments, the immunotherapy comprises an oncolytic virus. Oncolytic virus therapy utilizes a genetically modified virus (e.g., a herpes simplex virus, or other virus) to target cancer cells for programmed cell death via an immune response. An oncolytic virus is administered locally, e.g., injected into a tumor, where the virus enters the cancer cells and replicates. The replication can result in lysis of the cancer cells, resulting in the release of antigens and activating an immune response that targets the cancer cells for programmed cell death. Administration of the virus can be repeated until the desired effect is obtained (e.g., the tumor is eradicated). Oncolytic virus therapy (e.g., talimogene laherparepvec (Imlygic), or T-VEC) has been approved for treatment of melanoma.

In some embodiments, the immunotherapy comprises an engineered T cell. T cell therapy utilizes T cell that have been engineered to express an exogenous chimeric antigen receptor (CAR). As used herein, “chimeric antigen receptor” or “CAR” refers to an artificially constructed hybrid polypeptide comprising an antigen-binding domain (e.g., an antigen-binding portion of an antibody (e.g., a scFV)), a transmembrane domain, and a T-cell signaling and/or T-cell activation domain (e.g., intracellular signaling domain). CARs have the ability to redirect T-cell specificity and reactivity toward a selected tumor antigen in a non-MHC-restricted manner, exploiting the antigen-binding properties of monoclonal antibodies. Further discussion of CARs can be found, e.g., in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

As used herein, the term “tumor antigen” refers to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively.

### Immunotherapy-Associated Adverse Events

The term “adverse event” as used herein refers to any undesired effect that is caused by administration of an immunotherapy. An adverse event is considered to be caused by the immunotherapy if the adverse event occurs subsequent to the initiation of the immunotherapy. In general, the adverse event may be directly or indirectly caused by the immunotherapy.

Examples of adverse events that may be caused by immunotherapy include, without limitation, undesired immune responses, colitis, inflammation, skin toxicities including but not limiting to psoriatic, immunobullous, maculopapular, lichenoid, acantholytic eruptions, vitiligo, alopecias, vasculitides, and SJS/toxic epidermal necrolysis, neurotoxicity such as encephalitis, inflammatory arthritis, myocarditis, transverse myelitis, nephritis, myositis, Hepatotoxicity, Stevens-Johnson syndrome, Guillain-Barré syndrome, peripheral or autonomic neuropathy, pneumonitis, thrombocytopenia, and denous thromboembolism,

In some embodiments, the adverse event is immune-checkpoint inhibitor (ICI)-induced colitis.

### Treatment Methods

In one aspect, disclosed herein is a method of treating an immunotherapy-associated adverse event in a subject comprising (a) identifying the subject as having an elevated level of (soluble Major Histocompatibility Complex class I chain-related (sMIC) in a sample; (b) administering a corticosteroid to the subject. In some embodiments, the method further comprises identifying the subject has having an elevated level of IL-18 in the sample.

The methods may further comprise the step of comparing the level of sMIC (and optionally IL-18) in a sample from the patient to a predetermined criterion. In related embodiments, the method comprises detecting a level of sMIC (and optionally IL-18) that falls within a predetermined range indicative of corticosteroids being the therapy of choice for a patient experiencing an immunotherapy-associated adverse event (e.g., colitis). In some embodiments, the predetermined range of levels is higher than the levels of sMIC (and optionally IL-18) in healthy subjects.

The term “predetermined criterion” as used herein refers to a number indicative of the level of sMIC (and optionally IL-18) obtained from prior measurements of sMIC (and optionally IL-18) from biological samples (e.g., blood, tissue or feces) from a plurality of subjects not experiencing an immunotherapy-associated adverse event. In some variations, the predetermined criterion is the level of sMIC (and optionally IL-18) in healthy human controls (i.e., subjects with no clinical manifestation of cancer), in which case the level of sMIC (and optionally IL-18) determined in the method disclosed herein is increased compared to the level of sMIC (and optionally IL-18) sample obtained from the healthy controls. In some embodiments, the level of sMIC (and optionally IL-18) is increased by at least 2-fold (e.g., 2-fold, 3-fold, 4-fold, 5-fold, 6-fold, 7-fold, 8-fold, 9-fold, 10-fold, 11-fold, 12-fold, 13-fold, 14-fold, 15-fold, 20-fold or higher) compared to the level of sMIC (and optionally IL-18) in a sample obtained from the healthy controls.

In some variations, the predetermined criterion is the level of sMIC in healthy human controls (i.e., subjects with no clinical manifestation of an immunotherapy-associated event), in which case the level of sMIC determined in the method disclosed herein is decreased compared to the level of sMIC sample obtained from the healthy controls. In some embodiments, the level of sMIC is decreased by at least 2-fold (e.g., 2-fold, 3-fold, 4-fold, 5-fold, 6-fold, 7-fold, 8-fold, 9-fold, 10-fold, 11-fold, 12-fold, 13-fold, 14-fold, 15-fold, 20-fold or higher) compared to the level of sMIC in a sample obtained from the healthy controls.

The term “predetermined range” as used herein refers to a range of levels sMIC (and optionally IL-18) typically observed in human subjects experiencing an immunotherapy adverse event, in which case the level of sMIC (and optionally IL-18) is indicative of whether a patient will be responsive to treatment with a corticosteroid or treatment with a TNFα inhibitor or an integrin inhibitor if it falls within the predetermined range.

In other variations, the predetermined criterion or range might include information such as mean, standard deviation, quartile measurements, confidence intervals, or other information about the distribution or range of levels of sMIC (and optionally IL-18) in samples from subjects undergoing immunotherapy or subjects not undergoing immunotherapy. In still other variations, the predetermined criterion is a receiver operating characteristic curve based on data of levels sMIC (and optionally IL-18) in samples from subjects undergoing immunotherapy or subjects not undergoing immunotherapy. Optionally, the predetermined criterion is based on subjects further stratified by other characteristics that can be determined for a subject, to further refine the diagnostic precision. Such additional characteristics include, for example, sex, age, weight, smoking habits, race or ethnicity, blood pressure, other diseases, and medications.

To determine a measurement of MIC (and optionally IL-18) in a sample, the sample is contacted with a binding agent (e.g., antibody or antigen binding fragment thereof) that binds to MIC (or IL-18) for a time sufficient to allow immunocomplexes to form. Suitable MIC and IL-18 antibodies are commercially available. Immunocomplexes formed between the antibody and MIC (or IL-18) in the sample are then detected. The amount of MIC (or IL-18) in the sample is optionally quantitated by measuring the amount of the immunocomplex formed between the antibody and MIC (or IL-18). For example, the antibody can be quantitatively measured if it has a detectable label, or a secondary antibody can be used to quantify the immunocomplex. As demonstrated herein, detection of colon sMIC may be performed by immunohistochemistry (IHC).

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats can readily be adapted to employ the antibodies (or fragments thereof) of the present disclosure. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, FL Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985).

In some embodiments, an anti-MIC antibody, or antigen binding fragment thereof (or anti-IL-18 antibody or antigen binding fragment thereof) is attached to a solid support, and binding is detected by detecting a complex between the MIC (or IL-18) present in the sample and the antibody (or antigen binding fragment thereof) on the solid support. The antibody (or fragment thereof) optionally comprises a detectable label and binding is detected by detecting the label in the anti-MIC-antibody complex (or anti-IL-18-antibody complex).

Detection of the presence or absence of an anti-MIC-antibody complex (or anti-IL-18-antibody complex).complex be achieved using any method known in the art. For cell free binding assays, one of the components usually includes, or is coupled to, a detectable label. A wide variety of labels can be used, such as those that provide direct detection (such as radioactivity, luminescence, electrochemoluminescence, optical or electron density) or indirect detection (such as epitope tag such as the FLAG epitope, enzyme tag such as horseradish peroxidase). The label can be bound to the antibody, bispecific antibody, or incorporated into the structure of the antibody. A variety of methods can be used to detect the label, depending on the nature of the label and other assay components. For example, the label can be detected while bound to the solid substrate or subsequent to separation from the solid substrate. Labels can be directly detected through optical or electron density, radioactive emissions, nonradiative energy transfers or indirectly detected with antibody conjugates, or streptavidin-biotin conjugates.

Another example of a detection method is a reporter gene transcription assay, wherein a detectable product is produced upon binding of a MIC peptide interacting with an anti-MIC antibody (or binding of an IL-18 peptide interacting with an anti-IL-18 antibody). The detectable product may be observed by detecting e.g., β-galactosidase activity or luciferase activity.

In some embodiments, an elevated level of sMIC (and optionally also an elevated level of IL-18) identifies the subject as likely to respond to treatment with a corticosteroid. In this regard, subjects identified as having elevated levels of sMIC (and optionally also an elevated IL-18) relative to a predetermined criterion would be candidates for corticosteroid treatment instead of treatment with anti-TNFα.

The determining step of the methods described herein optionally comprises comparing the measurement of sMIC (and optionally IL-18) in a patient sample, and scoring the measurement from the sample as elevated (or decreased) based on statistical analysis or a ratio relative to the reference measurement. In some embodiments, the reference measurement comprises sMIC (and optionally IL-18) protein level in an arbitrary standard optionally further including statistical distribution information for the multiple measurements, such as standard deviation.

In some embodiments, the methods described herein comprise comparing the level of sMIC (and optionally IL-18) in a sample from the subject to the level of sMIC in a sample from a healthy subject, wherein an elevated level of sMIC (and optionally IL-18) compared to the predetermined criterion identifies the subject as a subject that would benefit from treatment with corticosteroids.

In some embodiments, the methods described herein comprise comparing the level of sMIC in a sample from the subject to the level of expression in a healthy subject, wherein an elevated level compared to the sample from the healthy subject and/or to the reference control set of patients identifies the subject as likely to benefit from treatment with corticosteroids.

In some embodiments, the methods described herein comprise comparing the level of sMIC in a sample from the subject to the level of expression in a healthy subject, wherein an elevated level compared to the sample from the healthy subject and/or to the reference control set of patients identifies the subject as likely to benefit from treatment with corticosteroids.

In some embodiments, the elevated level of sMIC in the sample comprises an amount that is ≥20 pg/mL. In some embodiments, the elevated level of sMIC in the sample comprises an amount ranging from ≥20 pg/mL to 100 ng/ml. In some embodiments, the elevated level of sMIC in the sample comprises an amount that is at least 20 pg/mL, or at least 25 pg/mL, or at least 30 pg/mL, or at least 35 pg/mL, or at least 40 pg/mL, or at least 45 pg/mL, or at least 50 pg/mL, or at least 55 pg/mL, or at least 60 pg/mL, or at least 65 pg/mL, or at least 70 pg/mL, or at least 75 pg/mL, or at least 80 pg/mL, or at least 85 pg/mL, or at least 90 pg/mL, or at least 95 pg/mL, or at least 100 pg/mL, or at least 1 ng/ml, or at least 10 ng/ml, or at least 20 ng/mL, or at least 30 ng/ml, or at least 40 ng/ml, or at least 50 ng/mL, or at least 60 ng/ml, or at least 70 ng/mL, or at least 80 ng/ml, or at least 90 ng/ml, or at least 100 ng/ml.

In some embodiments, the elevated level of sMIC in the sample comprises an amount that is ≥10% increase compared to baseline. In some embodiments, the elevated level of sMIC in the sample comprises an amount that is at least a 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or 100% increase compared to baseline.

In some embodiments, the elevated level of IL-18 in the sample comprises an amount that is ≥ (greater than or equal to) 90 pg/mL. In some embodiments, the elevated level of IL-18 in the sample comprises an amount that is ≥100 pg/mL. In some embodiments, the elevated level of IL-18 in the sample comprises an amount ranging from about 90 pg/mL to 3,000 pg/mL. In further embodiments, the elevated level of IL-18 in the sample comprises an amount ranging from about 100 pg/mL to 3,000 pg/mL. In some embodiments, the elevated level of IL-18 in the sample comprises an amount that is at least 90 pg/mL, or at least 100 pg/mL, or at least 150 pg/mL, or at least 200 pg/mL, or at least 300 pg/mL, or at least 400 pg/mL, or at least 500 pg/mL, or at least 600 pg/mL, or at least 700 pg/mL, or at least 800 pg/mL, or at least 900 pg/mL, or at least 1,000 pg/mL, or at least 1,500 pg/mL, or at least 2,000 pg/mL, or at least 2,500 pg/mL, or at least 3,000 pg/mL, or at least 3,500 pg/mL, or at least 4,000 pg/mL, or at least 4,500 pg/mL, or at least 5,000 pg/mL.

In some embodiments, the elevated level of IL-18 in the sample comprises an amount that is ≥10% increase compared to baseline. In some embodiments, the elevated level of IL-18 in the sample comprises an amount that is at least a 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or 100% increase compared to baseline.

The methods described herein may optionally comprise the step of identifying a subject as not being a candidate for treatment with an corticosteroid, if the level of sMIC in the blood sample from the subject is lower than the predetermined criterion.

In another aspect, disclosed herein is a method of treating an immunotherapy-associated adverse event in a subject comprising (a) detecting a decreased level of soluble Major Histocompatibility Complex class I chain-related (sMIC) in a sample; and (b) administering an anti-TNFα agent or an anti-integrin agent to the subject. In some embodiments, the methods further comprise the step of detecting a decreased level of IL-18 in the sample from the subject.

In some embodiments, the determining step comprises comparing the level of sMIC in a sample from the subject to the level of sMIC in a sample from a healthy subject, wherein a decreased level of sMIC compared to the predetermined criterion identifies the subject as a subject that would benefit from treatment with a TNFα inhibitor or an integrin inhibitor.

In some embodiments, the methods comprise (a) detecting in the sample from the subject the presence of (i) diagnostic markers of ulcerative colitis/irritable bowel syndrome diagnostic markers and (ii) a decreased level of sMIC; and, (b) administering a TNFα inhibitor to the subject.

In some embodiments, the methods comprise (a) detecting in the sample from the subject (i) an elevated level of sMIC (ii) a decreased level of sMIC and (ii) decreased level of IL-18; and, (b) administering a TNFα inhibitor to the subject.

In some embodiments, the TNFα inhibitor is etanercept, infliximab, adalimumab, certolizumab and golimumab. In some embodiments, the integrin inhibitor is an antibody (e.g. vedolizmab, etrolizumab, AMG-181), a small molecule (e.g. AJM300, CDP323), or a peptide (e.g. peptide X).

In some embodiments, the method comprises administering an IL-6R inhibitor to the subject. Exemplary IL-6R inhibitors include, but are not limited to, sarilumab and tocilizumab).

In some embodiments, the decreased level of sMIC in the sample comprises an amount that is ≤20 pg/mL. In some embodiments, the decreased level of sMIC in the sample comprises an amount that is less than 19 pg/mL, or less than 18 pg/mL, or less than 17 pg/mL, or less than 16 pg/mL, or less than 15 pg/mL, or less than 14 pg/mL, or less than 13 pg/mL, or less than 12 pg/mL, or less than 11 pg/mL, or less than 10 pg/mL, or less than 9 pg/mL, or less than 8 pg/mL, or less than 7 pg/mL, or less than 6 pg/mL, or less than 5 pg/mL, or less than 4 pg/mL ng/ml, or less than 3 pg/mL, or less than 2 pg/mL, or less than 1 pg/mL.

In some embodiments, the decreased level of sMIC in the sample comprises an amount that is ≥10% lower compared to baseline. In some embodiments, the decreased level of sMIC in the sample comprises an amount that is at least a 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or 100% lower compared to baseline.

In some embodiments, the decreased level of IL-18 in the sample comprises an amount that is <100 pg/mL. In some embodiments, the decreased level of IL-18 in the sample comprises an amount that is less than 100 pg/mL, or less than 90 pg/mL, or less than 80 pg/mL, or less than 70 pg/mL, or less than 60 pg/mL, or less than 50 pg/mL, or less than 40 pg/mL, or less than 30 pg/mL, or less than 20 pg/mL, or less than 10 pg/mL.

In some embodiments, the decreased level of IL-18 in the sample comprises an amount that is ≥10% decrease compared to baseline. In some embodiments, the decreased level of IL-18 in the sample comprises an amount that is at least a 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or 100% decrease compared to baseline.

As used herein, the term “treatment” refers to an approach for obtaining a beneficial or a desired result including, but not limited to, a therapeutic benefit or prevention of a condition, e.g., a side effect (such as an unwanted effect as described herein). The terms “treatment”, “treating”, and “ameliorating” are used interchangeably herein. In some embodiments, a therapeutic benefit is obtained by eradication or amelioration of the underlying disorder being treated. In some embodiments, a therapeutic benefit is obtained by reduction of, eradication, or amelioration of one or more of the symptoms, e.g., physiological symptoms, associated with the underlying disorder such that an improvement, e.g., change, is observed in the subject. In some embodiments, the subject can still be afflicted with the underlying disorder. In some embodiments, treatment comprises prevention of a condition, e.g., a side effect (such as an unwanted side effect from a therapy). Treatment or prevention of a condition or a side effect need not be a complete treatment or prevention of the condition or side effect.

In some embodiments, the sample is a serum sample, a tissue sample or a feces sample. In some embodiments, the tissues sample is a colon tissue sample.

In some embodiments, the determining step is performed between 1-14 days after administering an immunotherapy to the subject. In some embodiments, the determining step is performed 1, 2, 3, 4, 5, 6, 7, 8, 9, 10, 11, 12, 13, or 14 days after to administering the immunotherapy to the subject.

In some embodiments, the determining step is performed between within 24 hours after administering the immunotherapy to the subject. In some embodiments, the determining step is performed 30 minutes or 1, 2, 3, 4, 5, 6, 7, 8, 9, 10, 11, 12, 13, 14, 15, 16, 17, 18, 19, 20, 21, 22, 23 or 24 hours after administering the immunotherapy to the subject.

### Cancer

In any of the methods described herein, the subject is suffering from cancer. The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

In some embodiments, the cancer is a hematologic cancer. Examples of hematological (or hematogenous) cancers include, but are not limited to, leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the cancer is a solid tumor. Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include, but are not limited to, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein.

The technology described herein is further illustrated by the following examples which in no way should be construed as being further limiting.

### EXAMPLES

### Materials and Methods

Patients and samples: Patients with various types of cancer developed colitis after ICI treatment, as well as patients with ulcerative colitis (UC) were involved in this study. Colon biopsy samples from patients developed ICB therapy-induced diarrhea and colitis including left colon, sigmoid colon/rectum with or without histological (or microscopy) inflammation. Biopsy samples collected from inflamed and adjacent non-inflamed regions of the colon from the patients with UC. Immediately after collection, biopsy samples were fixed in 10% formalin solution for 24-48 h, stored in 70% of ethanol until embedded in paraffin.

Ipilimumab (αCTLA-4 IgG1) was administered by intravenous infusion every 3 weeks at a dose of 3 mg or 10 mg per kilogram of body weight. Patients could continue ipilimumab infusions with a maintenance treatment every 12 weeks in cases of tumour response or stable disease. Tremelimumab [αCTLA-4 IgG2] was administered intravenously every 4 weeks, at a dose of 3 mg or 10 mg per kilogram of body weight. Nivolumab and pembrolizumab [both αPD-1 IgG4] were administered intravenously every 2 and 3 weeks, at a dose of 3 mg and 2 mg per kilogram, respectively. Sociodemographic, clinical, biological, and endoscopic data were recorded using pre-specified case report forms. Colonic biopsy samples were collected to assess mRNA expression (NanoString nCount and DSP) and frequency of different immune cell population/sub-population

Immune gene mRNA expression measurement and analysis: The FFPE samples were sent to NanoString (Seattle, WA) to perform the mRNA extraction and mRNA level detection. In brief, RNA was extracted from 2× to 3×10 μm FFPE sections from the left colon region of the patients who developed diarrhea/colitis after ICI treatments, or from the inflamed region of the colon from the patients with UC. RNA extractions were performed with the Roche High Pure FFPE kit. Comprehensive immune gene expression levels were measured with both nCounter Autoimmune Discovery Consortium panel and Autoimmune Profiling panel (Nanostring Technologies, Inc.). Discovery panel contains 755 immune genes and 15 housekeeping genes, profiling panel contains 750 immune genes and 20 house-keeping genes, there are 255 genes were overlapped in these two panels. The assays were performed on the nCounter MAX system with 100 ng RNA per sample. The raw data was collected and then normalized for the positive control probes in each assay and also for the housekeeping genes in each panel with NanoString nSolver Analysis software v4.0. Differentially expressed genes were determined by “edgeR” 46 between ICI colitis group and ulcerative colitis (UC) group. The analysis was performed using normalized nCounter data in log 2 form, genes with FDR less than 0.05 and fold changes greater than 1.5 was defined as differentially expressed genes for ICI colitis and UC comparison. Volcano plot of differentially expressed genes was generated in R v.3.6. Principle Component Analysis (PCA) was conducted on mRNA expression levels from the colitis samples with command “prcomp” and illustrated with “ggbiplot” R packages. Gene set enrichment was performed using “GSVA” 47 with gene sets annotated by NanoString or from KEGG database to estimates pathway activity score on each sample in an unsupervised fashion. The gene sets/pathways were then hierarchically clustered according to their Euclidean distances. The heatmaps of standardized pathway scores were generated with “pheatmap” R packages.

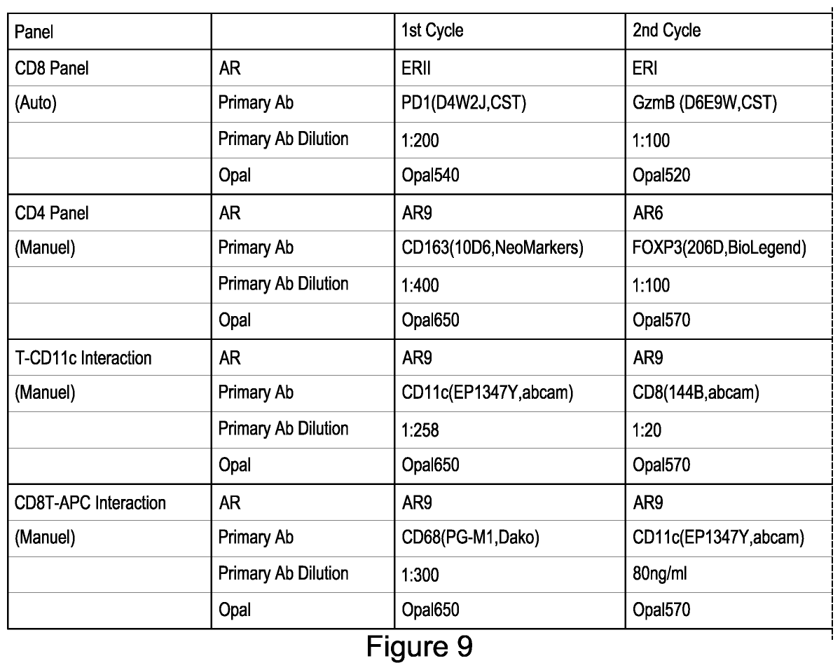

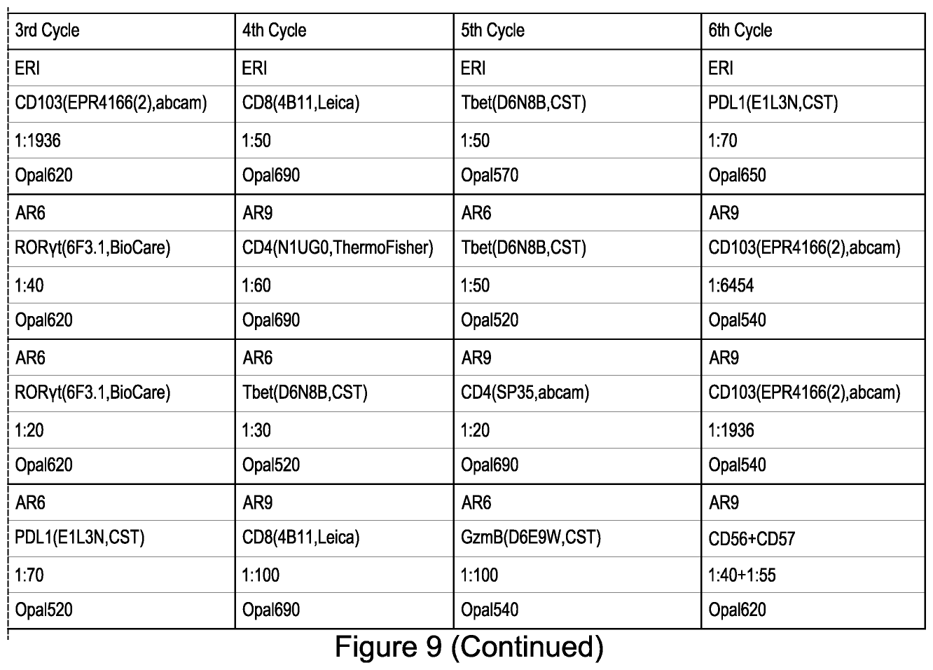

Quantitative multiplex immunohistochemistry (mIHC): For mIHC staining, 4 μm thick FFPE sections of the colon tissue was prepared. The antibodies used in all the panels were listed in FIG. 9. All the antibodies were validated on human colon sections using chromogen-based IHC before multiplex IHC. The multiplex IHC was performed using manual or automated Opal 7-color IHC kit (Akoya Biosciences, Menlo Park, CA). For manual experiments, the sections were deparaffinized, processed in antigen retrieval buffers (Akoya Biosciences) at 105° C. for 15 min, blocked for endogenous peroxidase with 3% H₂O₂ for 3 min followed by non-specific binding blocking at room temperature for 10 min, then incubated with primary antibodies for 30 min, followed by secondary antibody incubation for 10 min and the application of the Opal fluorophore for 10 min. The antibodies from the previous cycle were stripped with antigen retrieval solution at 105° C. for 15 min and the staining process was repeated for another antibody from 3% H₂O₂ incubation. The staining process was repeated until all the antibodies in the panel were completed. At last, all the sections were incubated with Spectral DAPI for 5 min, rinsed and then cover slipped with Prolong Gold Antifade Reagent (Invitrogen). Automated experiments were performed by Bond RX Stainer (Leica), the incubation time and process were almost identical to manual process except the antigen retrieval buffers were provided by Leica and the 3% H₂O₂ incubation steps were removed.

Multi-spectral images of the entire biopsy colon tissues from each region of all the patients were acquired on Vectra 3 slide scanner (Akoya Biosciences) and further analyzed with InForm software (version 2.4.2) for channel unmixing, cell segmentation and staining scoring. Thresholds for antibody positivity was determined and calibrated using representing images from all the colon sections performed in the same batch. The processed images were double checked for non-specific background, segmentation issue or other errors such as folded edges etc., the inappropriately processed images were excluded from quantitative analysis. Quantitative analysis was performed with Python and R package, including calculating cell frequencies of different phenotypes with specific marker or combination of markers against total cell number (DAPI+ cells); and adjacent neighborhood analysis, which was to calculate the frequency of a type of cells who were in close contact with (located adjacent to) another type of cells, defined as the distance between the centers of the adjacent cells was within the length of 12.5 μm (˜1× diameter of cells). Heatmap of cell types was generated using standard scores (z-scores) of the cell frequency obtained from quantification. All the statistical analysis (such as t-test) and plots were generated with R v.3.6.

Immunohistochemistry of MHC class I chain related protein (MIC): 4 μm thick FFPE sections of the colon tissue was cut from paraffin tissue blocks. After dewaxed and deparaffinized, the colon tissue slides were processed in antigen retrieval buffers AR6 (Akoya Biosciences) at 105° C. for 15 min, blocked for endogenous peroxidase with 3% H2O2 for 3 min followed by non-specific binding blocking with horse serum 10% at room temperature for 0.5 hour, then incubated with primary antibodies anti-human MIC A/B (Santa Cruz Biotechnology, Cat #sc-137242) at 0.2 μg/ml overnight at 4° C., followed by Vectastain Elite ABC kit (Vector Laboratories, Cat #PK6102) as instructed, and then developed with DAB chromogen (Vector Laboratories, Cat #SK-4105). The tissue sections were counterstained with Hematoxylin. The whole slides were scanned with Hamamatsu NanoZoomer 2.0-HT slide scanner. The MIC staining density in each colon region were semi-quantified using software ImageJ Fiji as previously described 48.

NanoString digital spatial profiling: 4 μm FFPE colon tissue sections from the patients who developed colitis after ICI treatments with either high and low MIC levels were selected, mounted on positively charged slide and sent to NanoString (Seattle, WA) to perform the Digital Spatial Profiling (DSP). In brief, the slide was stained with fluorescent morphology markers CD3-FITC, CD45-Texas Red and CD8a-Cy5 and then proceeded with DSP barcoded GeoMx Cancer Transcriptome Atlas (CTA) (>1800 RNA targets). Under the guidance of the morphology markers, segmented regions of interest (ROIs) were selected for either CD8 T cells population (CD8+, 15 ROIs total) or lymphocyte population without T cells (CD45+CD3−, 15 ROIs total) within about 660 μm×785 μm of tissue areas. The DSP barcode oligos corresponding to the CTA RNA targets within each ROI were then released by ultraviolet illumination to break the photocleavable linker and collected for NGS quantification. After sequencing QC and probe QC, probe counts were normalized against the 75th percentile of signal from their own ROI. Downstream differentially expressed genes were determined by edgeR between ROIs with high MIC staining and the ROIs with low MIC staining. The analysis was performed using normalized probe count data in log 2 form, genes with FDR less than 0.05 and fold changes greater than 1.5 was defined as differentially expressed genes. Volcano plot, PCA, heatmap of differentially expressed genes, hierarchical cluster were generated in R.v.3.6. Gene Set Enrichment (GSEA) was analyzed with Fast Gene Set Enrichment Analysis (“fgsea”) R package (v. 3.12) 49 to calculated the normalized enrichment score in the ROIs with high MIC staining compared with the ROIs with low MIC staining, the pathway annotations were provided by NanoString for the RNA targets in the CTA panel.

In vitro macrophage generation from peripheral monocytes: Human peripheral blood monocyte differentiation into macrophages. PBMCs from healthy donors were seeded at the density of 2×10⁶ cells/well in a 24 well plate with pre-warmed monocyte attachment media (MAM, Promocell, Cat. No. c-28051) and incubate at 37° C. After 1.5 hr incubation, non-adherent cells were removed. Adherent cells were washed three times with pre-warmed MAM before cultured in macrophage differentiation media respectively, the M1 macrophage generating media (Promocell c-28055) or the M2 macrophage generating media (Promocell c-28056). After 5 days of culture, 50% of the media were replaced with fresh media. At day 7, recombinant sMIC (rsMICB, his-tagged, SinoBiologicals Cat. No 10759-H08H) or control vehicle PBS were added to the culture at indicated concentration. The culture was continued for 2 days before being harvested for analyses.

Statistical Analysis: For quantitative multiplex IHC staining, all the statistical analysis including unpaired t-test, linear regression and correlation were performed with R v.3.6. p value of <0.05 was considered significant. GSEA analysis and the statistics was performed with “fgsea” package, the enriched pathways with a padj <0.05 was considered as significant.

### Example 1—ICI-Colitis Presented Unique and Shared Molecular/Cellular Signature with Ulcerative Colitis

To understand the inflammatory milieu of ICI-colitis as compared to ulcerative colitis (UCO, nanostring mRNA transcriptional profiling of formalin-fixed paraffin-embedded (FFPE) sections of colon biopsies from cohorts of cancer patients who received anti-PD1 monotherapy or combination therapy of anti-PD1 and anti-CTLA4 (ipilimumab) who developed symptoms indicative of potential ICI-colitis, and from UC patients was performed (Table 1).

TABLE 1
Clinical Information of Study Subjects.

| | | | Histologically | OS (yrs |
|---|---|---|---|---|
| Sample | | ICI | confirmed | since |
| No. | Cancer Type | therapy | colitis | biopsy) |
| 1 | Renal cell carcinoma | Combo | 0 | 3.4+ |
| 2 | Other | aPD1 | 0 | 0.3+ |
| 3 | Melanoma | Combo | 1 | 3.3+ |
| 4 | Renal cell carcinoma | Combo | 1 | 2.24+ |
| 5 | Melanoma | aPD1 | 1 | 3.1+ |
| 6 | Basal cell carcinoma | aPD1 | 1 | 3.0+ |
| 7 | Other | Combo | 1 | 2.5 |
| 8 | Cutaneous squamous cell | aPD1 | 1 | 2.2+ |
| | carcinoma | | | |
| 9 | Melanoma | Combo | 1 | 0.2 |
| 10 | Melanoma | aPD1 | 0 | 2.0+ |
| 11 | Renal cell carcinoma | Combo | 0 | 2.3+ |
| 12 | Melanoma | aPD1 | 0 | 2.3+ |
| 13 | Melanoma | Combo | 1 | 0.67 |
| 14 | NSCLC | aPD1 | 1 | 0.4 |
| 15 | Melanoma | Combo | 1 | 2.2+ |
| 16 | Melanoma | Combo | 1 | 1.1 |
| 17 | Renal cell carcinoma | Combo | 1 | 2+ |
| 18 | Head & Neck cancer | Combo | 1 | 1.7+ |
| 19 | Melanoma | Combo | 1 | 0.3 |
| 20 | Melanoma | aPD1 | 1 | 0.6+ |
| 21 | N/A | N/A | 1 | ND |
| 22 | N/A | N/A | 1 | ND |
| 23 | N/A | N/A | 1 | ND |
| 24 | N/A | N/A | 1 | ND |

Initial mRNA expression analyses with the universal Autoimmune Discovery Consortium (ARC) panel of five ICI-colitis colon samples and two active UC samples revealed the activation of genes involved in distinctly different immunological and inflammatory pathways in ICI-colitis, UC, and control colon (NC) with no histological evidence of colitis (data not shown). Specifically, ICI-colitis presented the active transcriptional profile related to antigen-processing and presentation and effector cell-mediated cytotoxic pathways, whereas UC presented the activation of genes related to NF-KB signaling and canonical IBD. Notably, a subset of ICI-colitis shares inflammatory signature, such as TNF signaling with UC (data not shown). Using inflammation-targeted Autoimmune RNA Profiling Panel (APP), further validating analyses of 13 ICI-colitis colon biopsies from nine combination therapy recipients and four anti-PD1 monotherapy recipients, four colon biopsies from active UC patients, and four colon biopsies from subjects who received ICI therapy but with no histological inflammation in the colon (NHC) was performed. The analyses confirmed both the distinct and the shared molecular featured of ICI-colitis and IBD/UC (data not shown) Specifically, one subset of ICI-colitis distinctively presented enriched gene signature of interferon signaling and immune cellular cytotoxicity, whereas the alternative subset of ICI-colitis distinctively presented enriched gene signatures associated with autoimmune IBD/UC, such as signaling pathways of TNFα, Th1/Th17, and IL-12. Noteworthy, no association of ICI-colitis subtypes with ICI treatment modality, whether combination therapy or anti-PD1/PDL1 monotherapy, was found.

Quantitative multiplex immunohistochemistry (mIHC) was subsequently performed, using the corresponding FFPE colon sections for APP RNA profiling, to assess whether colons of ICI-colitis and UC are enriched with key inflammatory cell types associated with the respective gene signatures. Unfailingly, colons of ICI-colitis presented one of the two distinct phenotypes, one predominantly enriched with granzymeB+ CD8 T cells and the other predominantly enriched with Th1/Th17 CD4 T cells, similar to UC colitis. Consistent with RNA profiling, the ICI-colitis phenotype did not associate with the regimen of single or combination of ICIs. Reactivation of colon tissue residual memory (TRM) CD8 T cells has been associated with ICI-colitis 19. We also observed an enrichment of granzymeB+ (Grzm B+) CD103+ tissue residual memory CD8 T cells (CD8TRM) in the colon of ICI-colitis as compared to IBD/UC (1f). However, Granzyme B+ CD8TRM only represents a small population of cytotoxic granzyme B+ CD8 T cells.

This Example will be repeated to assess the mRNA expression level of ULBP1-6 in colon biopsies of cancer patients receiving anti-PD1 monotherapy or combination therapy of anti-PD1 and anti-CTLA4 (ipilimumab) who developed symptoms indicative of potential ICI-colitis.

### Example 2—Accumulation of Soluble MIC in the Colon Distinguishes Molecular Pathogenic Subtypes of ICI-Colitis

Since human tumors, especially those that are advanced, shed soluble MIC (SMIC) as an immune evasion strategy, the presence of whether tumor-shed sMIC is associated with ICI-colitis in cancer patients was investigated.

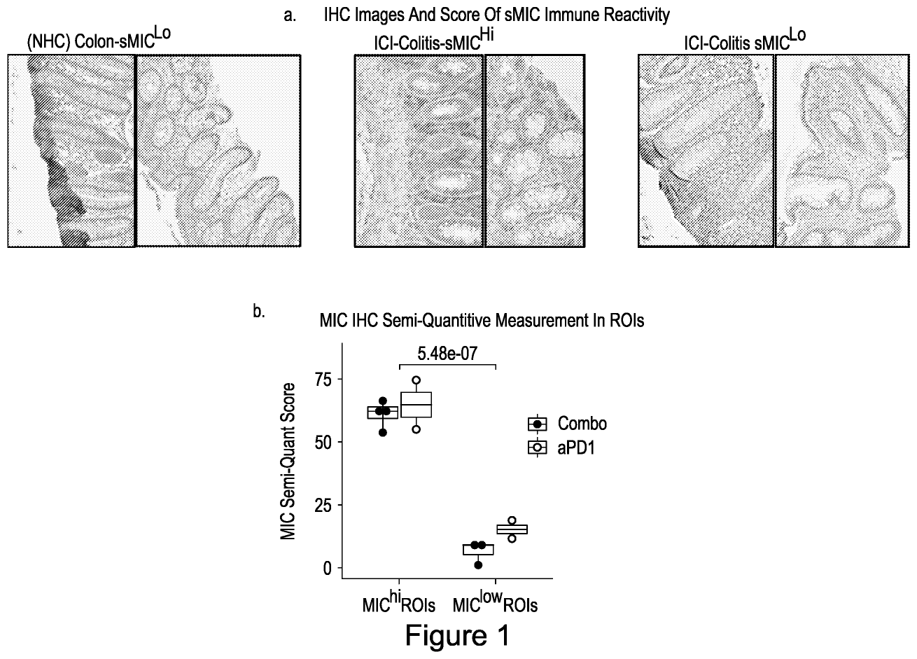

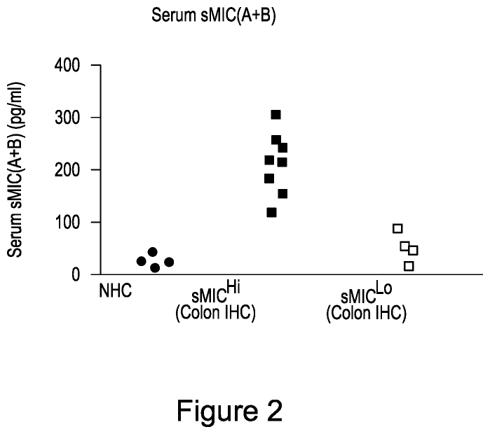

Colon sMIC-Hi defined by a staining intensity quantitative core equal or greater 50 when colon tissue was Immunohistochemistry stained with an anti-MIC-A/B antibody. The quantitation score was achieved with the software ImageJ Fiji as described by Crowe, A. R. & Yue, W. (Semi-quantitative Determination of Protein Expression using Immunohistochemistry Staining and Analysis: An Integrated Protocol. Bio Protoc 9, e3465, 2019). IHC evaluation demonstrated that 7 out of 11 ICI-colitis colon tissues exhibited strong anti-sMIC/MIC immune reactivity among the inflammatory cells of lamina propria of the mucosa, while the other four ICI-colitis mucosa exhibited weak anti-MIC immune reactivity (FIG. 1A and FIG. 1B). Noteworthy, mRNA levels of MIC-A or MIC-B did not associate with the level of anti-MIC/sMIC immune reactivity in the MICHi and MICLo mucosa, indicating that mucosa itself was not the source of MIC/sMIC. However, ELISA assay revealed that patients with MICHi mucosa also presented high levels of serum sMIC, suggesting that mucosal MIC is mostly the sMIC originated from tumor shedding or secretion into the circulation. All patients had elevated colon MIC IHC score had serum sMIC (A and B) greater than 90.4 pg/ml. For patients had low colon MIC IHC score, all had serum sMIC lower than 81 μg/ml, among whom, 6/7 had serum sMIC in the range of 3.78 pg/ml to 27.7 pg/ml. See FIG. 2.

To understand the impact of presence of sMIC in mucosa on ICI-colitis pathophysiology and the inflammatory milieu, Digital Spatial Profiling (DSP) was performed at the mRNA level of CD3+CD8+ T cells and CD45+CD3− myeloid cell types presented in the inflamed regions of interest (ROI) that respectively exhibited high and low immunoreactivity with the anti-sMIC/MIC antibody. FFPE sections of representative MIC^{Hi} and MIC^{Lo} colons from patients who received anti-PD1 and anti-CTLA4 combination therapy or anti-PD1 monotherapy were subjected to the DSP analyses. Remarkably, both CD8 T cells and CD45+CD3-myeloid cells presented significantly differential gene expression pattern in the inflamed sMIC^{Hi} vs. sMIC^{Lo} mucosa as demonstrated by volcano plots, principal component analyses (PCA), and gene clustering heatmap (data not shown), irrespective of anti-PD1 monotherapy or anti-PD1/anti-CTLA4 regimen. Gene Set Enrichment analyses (GSEA) revealed markedly enriched gene expression pathways relevant to effector cell activation and antigen presentation activities in sMIC^{Hi} mucosa in CD8 T cell subsets as well in CD45+CD3− myeloid compartment (data not shown). These initial findings indicated a different inflammatory landscape in sMIC^{Hi} and sMIC^{Lo} ICI-colitis mucosa.

This Example will be repeated to determine whether the amount (i.e., increased or decreased expression) of ULBP1-6 is associated with a different inflammatory landscape with respect to ICI-colitis in cancer patients who received anti-PD1 and anti-CTLA4 combination therapy or anti-PD1 monotherapy.

### Example 3—Enrichment of Heterogenous Cytotoxic CD8 T Cells in sMIC^{Hi} ICI-Colitis Mucosa

Gene enrichment analyses of CD8 T cell gene signature in the inflamed mucosa revealed a marked enrichment of genes of effector cytotoxic function, such as granzyme A/B and perforin (PFR1) in sMICHi ICI-colitis mucosa, with the most highly expressed in the mucosa of combination therapy. These CD8 T cells demonstrated high expression levels of the proliferation marker MK167, minimal levels of BCL2 expression indicating the potential of high apoptotic, low levels of IL-2 or IL-2R, indicating a high potential of high turnaround or contraction of these CD8 T cells. This population of CD8 T cells also demonstrated markedly high expression levels of the activation markers, HLA-DRs, CD38, and CD69. These markers signify a newly activated effector CD8 T cell population in the sMIC^{Hi} ICI-colitis mucosa, which is most pronounced with anti-PD1 and anti-CTLA4 combination therapy. Interestingly, a high expression level of the co-stimulatory molecules CD86/80 was also present, suggesting a potential activation of HLA-DR+BCL-2-CD25low/negativeFashi regulatory CD8 T (CD8Treg) cells.

Conversely, an array of immune checkpoint molecules, including CTLA4 and PD-1 was significantly increased on CD8 T cells from sMIC^{Lo} ICI-colitis mucosa (data not shown), implicating a potential immune checkpoint inhibition-induced activation. Notably, there is remarkably higher levels of IL-12R/IL-23R and IL-2R expression on CD8 T cells in sMIC^{Lo} ICI-colitis mucosa, suggesting a potential contribution of signaling by IL-12/IL-23 canonical autoimmune inflammatory pathway to therapy induced colon pathology in this subpopulation of patients.

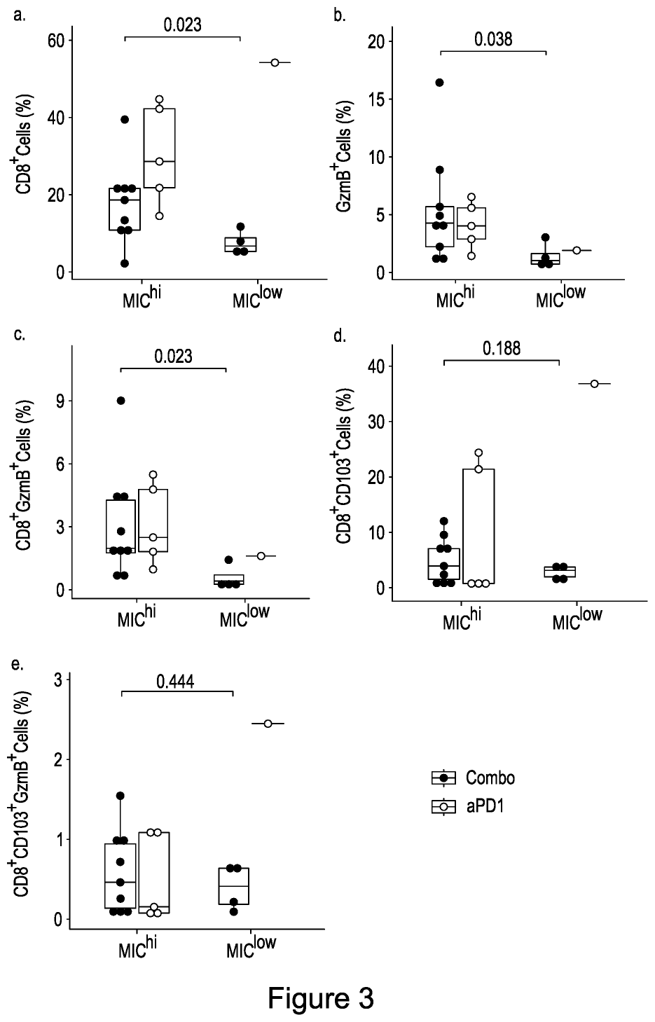

To validate the data of RNA expression profiling that sMIC^{Hi} ICI-colitis is associated with CD8 T cell granzyme B cytotoxic activity, quantitative mIHC analyses with relative marker of cytotoxic CD8 T cells was performed. There is a significantly higher density of CD8 T cells in the inflamed sMIC^{Hi} ICI-colitis mucosa than in the inflamed sMIC^{Lo} ICI-colitis mucosa in patients received combination therapy (FIG. 3A). The numbers of granzyme B+ cells and granzyme B+ CD8 T cells in the inflamed region of sMIC^{Hi} mucosa were significantly higher with anti-CTLA4 and anti-PD1 combination therapy than in the inflamed region of sMIC^{Lo} mucosa (FIGS. 3B and 3C). With anti-PD1 monotherapy, a trend of higher numbers of granzyme B+ cells and granzyme B+CD8 T cells was seen in the inflamed region of sMIC^{Hi} mucosa than in the sMIC^{Lo} mucosa; however, the statistical significance cannot be assessed due to the small population size of sMIC^{Lo} patients (FIGS. 3B and 3C). This observation agrees with the gene signature presenting a potential contribution CD8 T cell granzyme B activity to ICI-colitis in sMIC^{Hi} patients. Notably, no significant differences in granzyme B+ CD103⁺CD8⁺ T cells or the density of CD103⁺CD8⁺ TRM was observed between the sMIC^{Hi} ICI-colitis and sMIC^{Hi} ICI-colitis inflamed mucosa (FIGS. 3D and 3E).

This Example will be repeated to determine whether there is an association between elevated levels of ULBP1-6 and granzyme B cytotoxic activity in a sample from an ICI-colitis patient.

### Example 4—Signature of IBD-UC Inflammatory Pathways in the Mucosa of sMIC^{Lo} ICI-Colitis

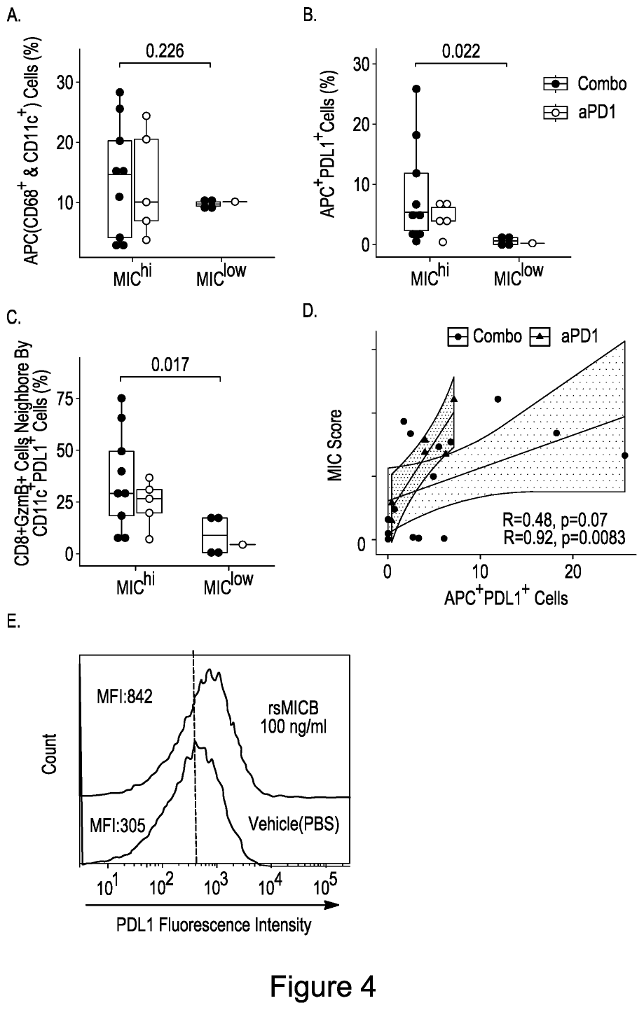

To further understand the inflammatory milieu in the sMIC^{Hi} ICI-colitis and sMIC^{Lo} ICI-colitis, the gene signatures of CD45⁺CD3⁻ myeloid cells was analyzed. A significant increase in DC maturation markers CD40/80/86 presented in the mucosal CD45⁺CD3⁻ myeloid cells of sMIC^{Hi} ICI-colitis (data not shown), which likely contributed to the increased co-stimulation of CD8 T cells and thus activation. RELB1, a gene encoding a protein that represses DC maturation (Shih et al., Nat Immunol 13, 1162-1170, 2012), was significantly reduced in sMIC^{Hi} ICI-colitis, strengthening the evidence of increased DC maturation in the inflamed mucosa. The increased expression of FAS, an inducer of DC maturation in the absence of IL-12 signaling²⁵, further supports the observation. Most interestingly, PDL1 (CD274) expression on CD45+CD3-myeloid-prominent cells was significantly higher in inflamed sMIC^{Hi}-ICI mucosa than in the inflamed sMIC^{Lo}-ICI mucosa, which is most pronounced with combination therapy (FIG. 4).

RNA expression of CD45+CD3-myeloid cells in the sMIC^{Lo} ICI-colitis mucosa was dominated by the signature of canonical IBD/UC inflammatory cytokines, exemplified by enriched expression of genes related to IL-22, TNF, IL-12/23, IL-17, and IL-6 signaling. These cytokines can form an inflammatory milieu to directly activate CD8 TRM via IL-12 signaling^{26,27} and to facilitate the differentiation of CD4 T cells into Th1/Th17 subtypes^{28,29}. This is consistent with the observation of increased IL-12R/II-23R on CD8 T cells in the SMIC^{Lo} ICI-colitis mucosa (data not shown). These gene expression pattern suggest that, in contrast to enhanced maturation of antigen-presenting cells (APCs) and antigen-presentation capacity to activate effector cytotoxic CD8 T cells in sMIC^{Hi}-ICI mucosa, amplified canonical IBD/UC inflammatory signaling may be a major driver of sMIC^{Lo} ICI-colitis.

To corroborate the concept that an induction of PD-L1 on APCs in part contributes to anti-PD1-induced ICI-colitis in sMIC^{Hi} mucosa, the spatial relationship of granzymeB+ CD8 T cells with PD-L1+ APCs was assessed by IHC in sMIC^{Hi} and sMIC^{Lo} mucosa. Although there is no significant difference in total density of APCs in the inflamed region of sMIC^{Hi} vs. sMIC^{Lo} ICI-colitis mucosa (FIG. 4A), there was a significantly higher number of PDL1+APCs and close spatial proximity of PDL1+APCs to granzymeB+ CD8 T cells in the sMIC^{Hi} ICI-colitis mucosa in patients received anti-PD1 and anti-CTLA4 combination therapy (FIGS. 4B and 4C). Due to the small population of patients in the sMIC^{Lo} group who developed ICI-colitis upon anti-PD1 monotherapy, a statistical significance cannot be assessed although there is a trend of a higher number of APCs expressing PDL1 and a higher number of granzymeB+ CD8 T cells in close spatial proximity with PDL1+APCs in patients with sMIC^{Hi} mucosa (FIG. 4C). A significant correlation of sMIC immune reactivity score with the number of PDL1+ APCs was found in the ICI-colitis mucosa (FIG. 4D). In vitro assay confirmed that sMIC can induce PDL1 expression on human peripheral monocyte differentiated M1 macrophages (FIG. 4E). Together, these observations indicate a possible mediating anti-PD1 or anti-PD1/anti-CTLA4 combination therapy-induced CD8 cytotoxic T cell activation and ICI-colitis in sMIC^{Hi} patients through sMIC induction of PDL1 expression on APCs.

### Example 5—Mucosal sMIC and Associated ICI-Colitis Molecular Signature Uncouples Clinical Response to Corticosteroid Management

Review of ICI-colitis incidence revealed that colon sMIC^{Lo} patients had less incidence (7 out 11, 64%) of ICI-colitis than colon sMIC^{Hi} patients (7/7, 100%). Intriguingly, the data demonstrates that colon sMIC immune reactivity correlates with patient's response to corticosteroids. Colon sMIC^{Hi} patients are highly responsive to corticosteroid management ( 5/7, 71.4%), whereas only a small percentage of colon sMIC^{Lo} patients responded to corticosteroid ( 2/7, 28.5%). In both colon sMIC^{Hi} and sMIC^{Lo} patient, steroid failures are all responsive to the 2nd line management, one or two doses of infliximab (anti-TNFα) or occasional one dose of vedolizumab (anti-α4β7).

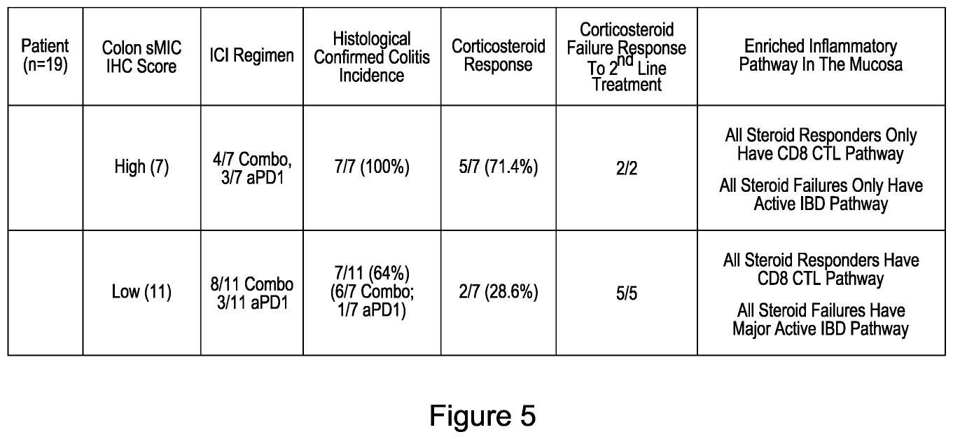

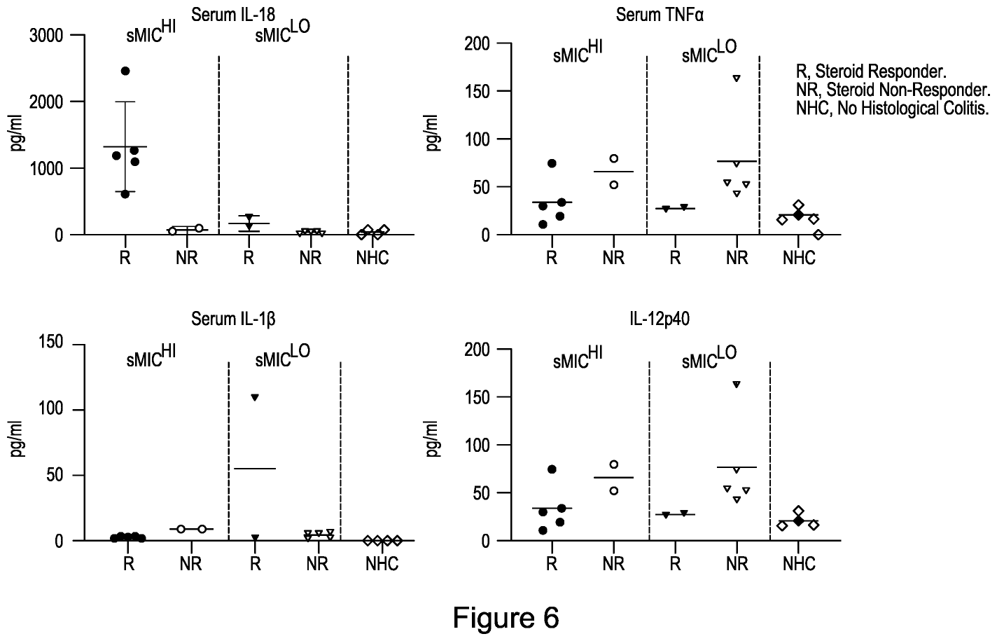

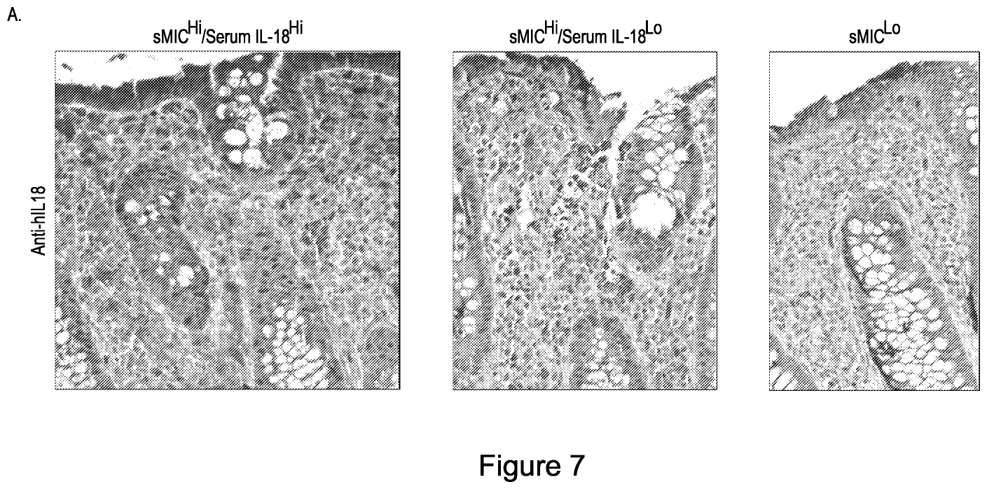

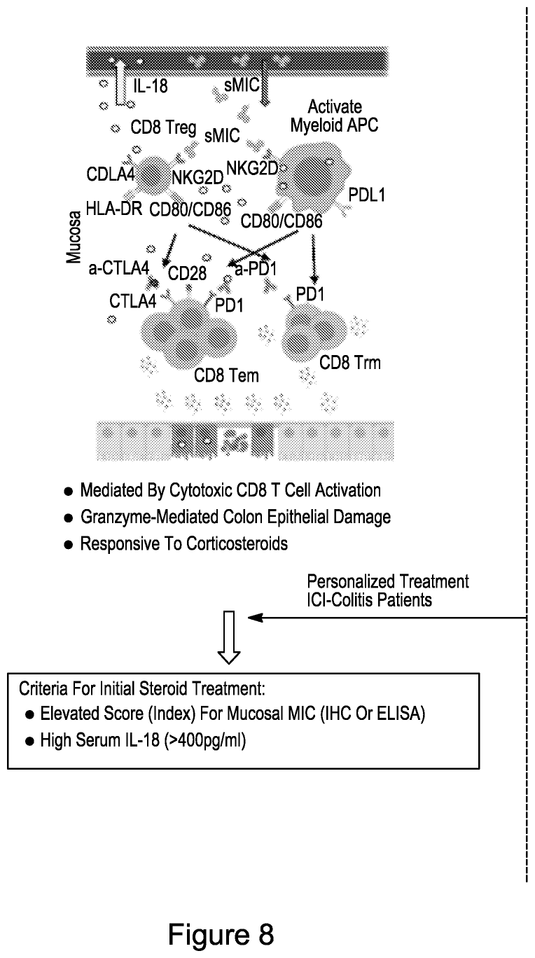

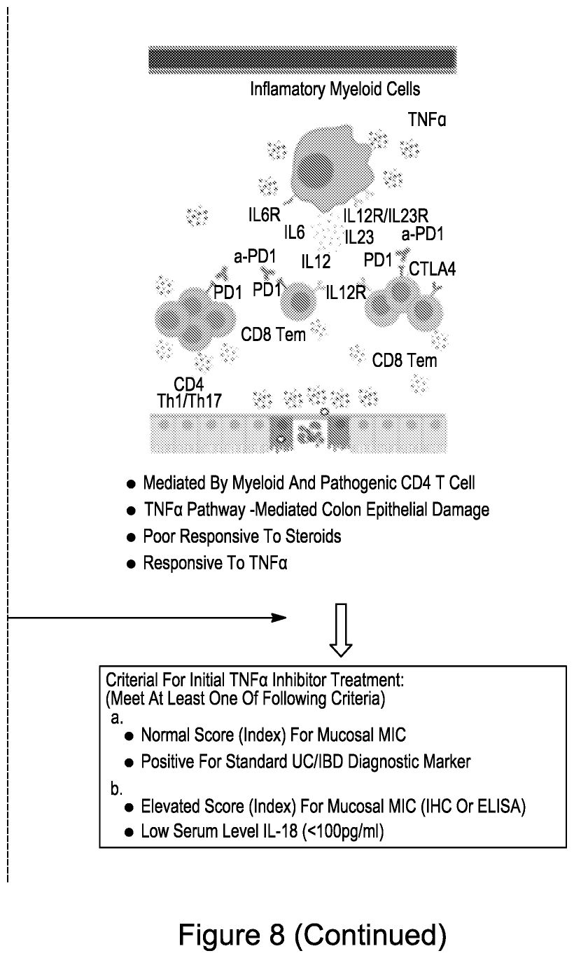

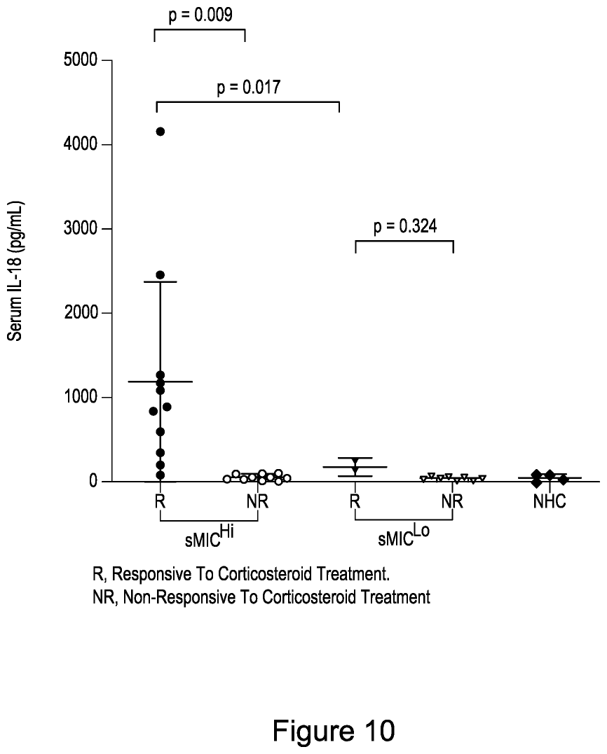

Data from RNA profiling of the colon and mIHC revealed that all patients that were responsive to steroid had high levels of serum IL-18. Using the 42-PLEX cytokine array assay at the service of Eve Technologies, the range of serum IL-18 in patients that did not develop colitis is 0-91.0 pg/ml as detected, the range of patients who have elevated colon or serum sMIC but did not respond to corticosteroids are within the range of 0-91 pg/ml. Instead, the range of patients with elevated colon or serum sMIC and responded to corticosteroids all had a serum value of IL-18 greater than 400 pg/ml. Noteworthy, all the corticosteroid responders ( 5/7) in the sMICHi patients had significantly higher levels of serum IL-18 (FIG. 5). Re-analyses of the RNA profiling of the colon biopsies revealed an enriched expression of genes involved in inflammasome pathways, including IL-18 and Caspase 1, in the sMICHi and corticosteroid-responsive patients as compared to other patients (data not shown). On the contrary, all patients that were steroid failure had enriched molecule signature of TNFα/Th17/IL-12 cytokine signaling, with high similarity to autoimmune UC/IBD^{30,31}. Collectively, these data demonstrate that the ICI-colitis responsiveness to the 1st line corticosteroid management could potentially be identified by colonic sMIC and cytokine IL-18 signatures (FIG. 6). A follow up study was performed to determine serum levels of IL-18 in additional cancer patients who received immune checkpoint inhibitor (ICI)-therapy. See FIG. 10. The data demonstrated that sMICHi ICI-colitis patients who are responsive to corticosteroids have significantly higher levels of serum IL-18 relative to sMICHi ICI-colitis patients who are not responsive to corticosteroids.

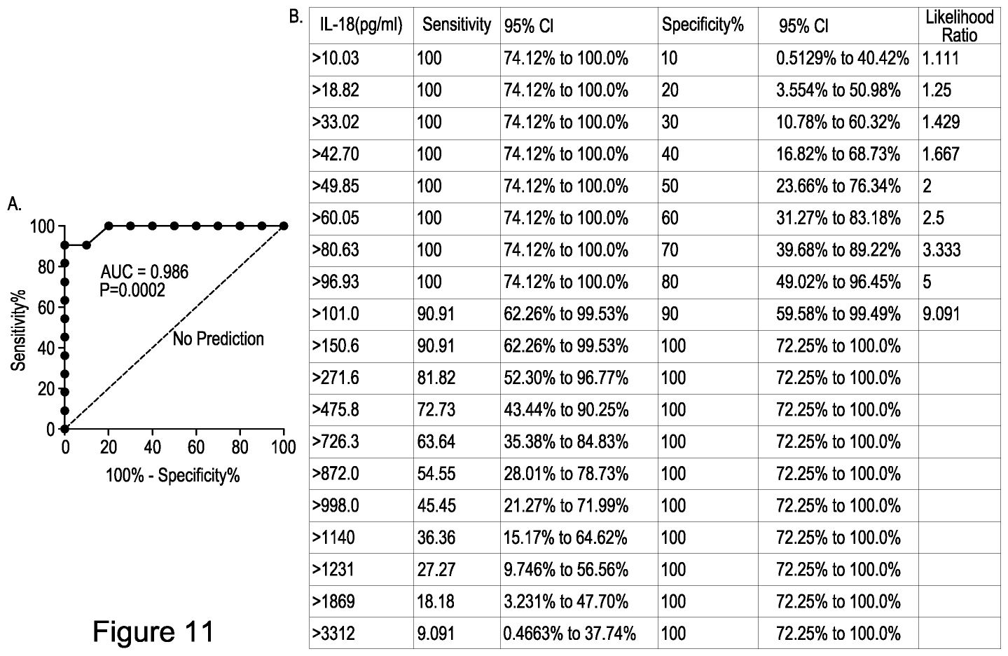

A statistical analysis was performed to investigate the predictive ability of serum IL-18 in sMICHi ICI-colitis patients' response to corticosteroid treatment. See FIG. 11. FIG. 11 shows the sensitivity and specificity of serum levels of IL-18 in predicting sMIChI ICI-colitis patient's response to corticosteroid. With serum level IL-18 of 150.6 pg/ml, a sensitivity of 90.90% and specificity of 100% is reached. With serum levels of IL-18 of 101.0 pg/ml, a sensitivity of 90.90% and specificity of 90% is reached. sMICHi patients defined as colon sMIC immunohistochemistry Staining (IHC) score by Image J quantification criteria of 33 or greater and or serum levels of sMIC (combination of sMICA and sMICB) of 80 pg/ml.

This Example will be repeated to determine whether elevated levels of ULBP1-6 in a sample an ICI-colitis patient correlates with the patient's responsiveness to corticosteroid treatment.

### Discussion:

In this study, inflammatory molecular signatures and pathways in ICI-colitis that could ultimately guide ICI-colitis management was explored. It was determined that ICI-colitis manifested two pathogenic pathways which displayed differential clinical response to corticosteroids. One pathogenic pathway was associated with activation of CD8 T cell granzyme cell cytotoxicity which was linked to responsiveness to corticosteroid immune suppressants. The alternative pathway was associated with amplified signaling of TNFα, Th1/Th17, and IL-12, all of which are canonical signaling pathways seen in UC32-34, in whom the ICI-colitis failed to respond to steroid management. Intriguingly, the two pathogenic pathways can be identified by accumulation of tumor-derived sMIC in the colon and early inflammatory microenvironment similar to autoimmune UC/IBD. Importantly, the data provided herein shows that ICI-colitis patients with high abundance of mucosal sMIC and high levels of serum IL-18 were steroid responders; conversely, majority of the ICI-colitis patients with low or no abundance of mucosal sMIC did not respond to steroids, but typically responded well to 2nd line management with TNF inhibitor or vedolizumab.

Currently, corticosteroids are the recommended first line treatment of ICI-colitis while the TNFα inhibitor infliximab and the α4β7 inhibitor vedolizumab are recommended as the second line treatment for patients who have failed corticosteroids⁷. The immune suppressive effect of corticosteroids is primarily through inhibiting the proliferation of naïve and newly activated effector CD8 T cells and down regulation of granzyme B expression³⁵⁻³⁹. Consistent with these mechanistic understandings, the data provided herein shows that nearly all steroid responsive ICI-colitis patients shared the molecular and cellular signatures of activated cytotoxic CD8 T cells in the colon. Conversely, most steroid non-responsive ICI-colitis patients were deficient of the signature of activated cytotoxic CD8 T cells but demonstrated dominant cytokine signaling signature of IBD/UC. The features of newly activated cytotoxic CD8 T cells, presented with high proliferation potential, high cytotoxicity, and high expression of activation markers, were markedly enriched in ICI-colitis mucosa with high abundance of sMIC. On the contrary, ICI-colitis mucosa with low abundance of sMIC did not display features of newly activated cytotoxic CD8 T cells but displayed molecular features of UC/IBD, such as amplified TNFα/Th17/IL-12 cytokine signaling. The data provided herein suggests a potential differentiation of ICI-colitis patients who will or will not benefit from initial steroid treatment by colonic sMIC abundancy and UC/IBD cytokine profiles.

High levels of circulating sMIC have been correlated with poor clinical outcome of ICI therapy^{41,42}. Surprisingly, the current study found that high levels of sMIC in the ICI-colitis mucosa correlated with the signature of high CD8 T cell cytotoxicity with immune checkpoint blockade therapy in corticosteroid responsive patients. The elevated levels of serum IL-18 and enriched IL-18/CASP1 expression profiles in the colon may conceivably provide an explanation of increased CD8 T cell cytotoxicity in the presence of sMIC, as IL-18 has been shown to be able to restore NKG2D expression and cytotoxicity of NK cells through activation of JNK pathways in the presence of immune suppressive factors⁴³. Moreover, PDL1 expression is increased on antigen presenting cells in association with sMIC, which offers an additional potential mechanistic explanation of increased CD8 T cell cytotoxic activity when the PD1/PDL1 inhibitory pathway is interrupted. Surprisingly, a signature of the Ki67+ BCL-2-FAS+ fast turnaround HLA-DR+ regulatory CD8 T cells was predominant in sMIC^{Hi} ICI-colitis mucosa with the combination therapy.

The current guidelines to administer corticosteroids as the first line treatment of ICI-colitis is based upon empiric clinical experience and adaption from the UC/IBD experience⁷. However, a scientific basis to support this clinical strategy is lacking. Under current guidelines, controlling ICI-colitis with the second line immune modulator, such as anti-TNFα (infliximab) or anti-α4β7 (vedolizumab), is delayed and delegated to patients who failed to respond to steroids. While the impact of low-dose corticosteroids on ICI efficacy is unequivocal 10, high-dose corticosteroids may mitigate ICI therapy⁴⁴. Nevertheless, severe toxicities such as colitis can complicate and undermine clinical benefits of ICI therapy by delaying the resumption or even discontinuation of the ICI therapy. Collectively, these combined factors could have a negative impact on the clinical course of cancer patients with ICI-colitis^{8,10,45}. The data provided herein allows for patient selection by assessing colon MIC/sMIC immune reactivity, and levels of IL-18 (and optionally established UC/IBD diagnostic markers) to select a subpopulation of patients for initial targeted anti-TNFα instead of corticosteroid treatment to rapidly control ICI-colitis and allow for an earlier resumption of ICI therapy.

### REFERENCES

1. Postow, M. A., Sidlow, R. & Hellmann, M. D. Immune-Related Adverse Events Associated with Immune Checkpoint Blockade. New England Journal of Medicine 378, 158-168 (2018).
2. Friedman, C. F., Proverbs-Singh, T. A. & Postow, M. A. Treatment of the Immune-Related Adverse Effects of Immune Checkpoint Inhibitors: A Review. JAMA Oncol 2, 1346-1353 (2016).
3. Spain, L., Diem, S. & Larkin, J. Management of toxicities of immune checkpoint inhibitors. Cancer Treat Rev 44, 51-60 (2016).
4. Khoja, L., Day, D., Wei-Wu Chen, T., Siu, L. L. & Hansen, A. R. Tumour- and class-specific patterns of immune-related adverse events of immune checkpoint inhibitors: a systematic review. Ann Oncol 28, 2377-2385 (2017).
5. Kumar, V., et al. Current Diagnosis and Management of Immune Related Adverse Events (irAEs) Induced by Immune Checkpoint Inhibitor Therapy. Front Pharmacol 8, 49 (2017).
6. Postow, M. A., Sidlow, R. & Hellmann, M. D. Immune-Related Adverse Events Associated with Immune Checkpoint Blockade. N Engl J Med 378, 158-168 (2018).
7. Brahmer, J. R., et al. Society for Immunotherapy of Cancer (SITC) clinical practice guideline on immune checkpoint inhibitor-related adverse events. J Immunother Cancer 9 (2021).
8. Drakaki, A., et al. Association of systemic corticosteroids with overall survival in patients receiving cancer immunotherapy for advanced melanoma, non-small cell lung cancer or urothelial cancer in routine clinical practice. Annals of Oncology 30, xi16-xi17 (2019).
9. Martins, F., et al. Adverse effects of immune-checkpoint inhibitors:
   epidemiology, management and surveillance. Nature Reviews Clinical Oncology 16, 563-580 (2019).
10. Petrelli, F., et al. Association of steroids use with survival in patients treated with immune checkpoint inhibitors: a systematic review and meta-analysis. Cancers 12, 546 (2020).
11. Brahmer, J. R., et al. Management of Immune-Related Adverse Events in Patients Treated With Immune Checkpoint Inhibitor Therapy: American Society of Clinical Oncology Clinical Practice Guideline. J Clin Oncol 36, 1714-1768 (2018).
12. Puzanov, I., et al. Managing toxicities associated with immune checkpoint inhibitors: consensus recommendations from the Society for Immunotherapy of Cancer (SITC) Toxicity Management Working Group. Journal for Immuno Therapy of Cancer 5, 95 (2017).
13. Thompson, J. A. New NCCN Guidelines: Recognition and Management of Immunotherapy-Related Toxicity. J Natl Compr Canc Netw 16, 594-596 (2018).
14. Abu-Sbeih, H. & Wang, Y. Management Considerations for Immune Checkpoint Inhibitor-Induced Enterocolitis Based on Management of Inflammatory Bowel Disease. Inflamm Bowel Dis 26, 662-668 (2020).
15. Abu-Sbeih, H., Ali, F. S. & Wang, Y. Immune-checkpoint inhibitors induced diarrhea and colitis: a review of incidence, pathogenesis and management. Curr Opin Gastroenterol 36, 25-32 (2020).
16. Bergqvist, V., et al. Vedolizumab treatment for immune checkpoint inhibitor-induced enterocolitis. Cancer Immunol Immunother 66, 581-592 (2017).
17. Powell, N., et al. British Society of Gastroenterology endorsed guidance for the management of immune checkpoint inhibitor-induced enterocolitis. Lancet Gastroenterol Hepatol 5, 679-697 (2020).
18. Chen, A. Y., Wolchok, J. D. & Bass, A. R. TNF in the era of immune checkpoint inhibitors: friend or foe? Nat Rev Rheumatol 17, 213-223 (2021).
19. Sasson, S. C., et al. IFNγ-producing CD8+ tissue resident memory T cells are a targetable hallmark of immune checkpoint inhibitor-colitis. Gastroenterology (2021).
20. Perez-Ruiz, E., et al. Prophylactic TNF blockade uncouples efficacy and toxicity in dual CTLA-4 and PD-1 immunotherapy. Nature 569, 428-432 (2019).
21. Liu, G., et al. Perturbation of NK cell peripheral homeostasis accelerates prostate carcinoma metastasis. The Journal of clinical investigation 123, 4410-4422 (2013).
22. Machicote, A., Belén, S., Baz, P., Billordo, L. A. & Fainboim, L. Human CD8+HLA-DR+ Regulatory T Cells, Similarly to Classical CD4+Foxp3+ Cells, Suppress Immune Responses via PD-1/PD-L1 Axis. Frontiers in Immunology 9 (2018).
23. Saraiva, D. P., Jacinto, A., Borralho, P., Braga, S. & Cabral, M. G. HLA-DR in Cytotoxic T Lymphocytes Predicts Breast Cancer Patients' Response to Neoadjuvant Chemotherapy. Frontiers in Immunology 9 (2018).
24. Shih, V. F., et al. Control of RelB during dendritic cell activation integrates canonical and noncanonical NF-kappaB pathways. Nat Immunol 13, 1162-1170 (2012).
25. Rescigno, M., et al. Fas engagement induces the maturation of dendritic cells (DCs), the release of interleukin (IL)-1beta, and the production of interferon gamma in the absence of IL-12 during DC-T cell cognate interaction: a new role for Fas ligand in inflammatory responses. J Exp Med 192, 1661-1668 (2000).
26. Xiao, Z., Casey, K. A., Jameson, S. C., Curtsinger, J. M. & Mescher, M. F. Programming for CD8 T Cell Memory Development Requires IL-12 or Type I IFN. The Journal of Immunology 182, 2786-2794 (2009).
27. Molodtsov, A. & Turk, M. J. Tissue Resident CD8 Memory T Cell Responses in Cancer and Autoimmunity. Frontiers in Immunology 9 (2018).
28. Trinchieri, G. Interleukin-12 and the regulation of innate resistance and adaptive immunity. Nat Rev Immunol 3, 133-146 (2003).
29. Xu, M., et al. Regulation of antitumor immune responses by the IL-12 family cytokines, IL-12, IL-23, and IL-27. Clin Dev Immunol 2010 (2010).
30. Chen, M. L. & Sundrud, M. S. Cytokine Networks and T-Cell Subsets in Inflammatory Bowel Diseases. Inflamm Bowel Dis 22, 1157-1167 (2016).
31. Friedrich, M., Pohin, M. & Powrie, F. Cytokine Networks in the Pathophysiology of Inflammatory Bowel Disease. Immunity 50, 992-1006 (2019).
32. Imam, T., Park, S., Kaplan, M. H. & Olson, M. R. Effector T Helper Cell Subsets in Inflammatory Bowel Diseases. Frontiers in immunology 9, 1212-1212 (2018).
33. Tindemans, I., Joosse, M. E. & Samsom, J. N. Dissecting the Heterogeneity in T-Cell Mediated Inflammation in IBD. Cells 9, 110 (2020).
34. Schurich, A., Raine, C., Morris, V. & Ciurtin, C. The role of IL-12/23 in T cell-related chronic inflammation: implications of immunodeficiency and therapeutic blockade. Rheumatology 57, 246-254 (2017).
35. Acharya, N., et al. Endogenous Glucocorticoid Signaling Regulates CD8(+) T Cell Differentiation and Development of Dysfunction in the Tumor Microenvironment. Immunity 53, 658-671 e656 (2020).
36. Giles, A. J., et al. Dexamethasone-induced immunosuppression: mechanisms and implications for immunotherapy. J Immunother Cancer 6, 51 (2018).
37. Taves, M. D. & Ashwell, J. D. Glucocorticoids in T cell development, differentiation and function. Nat Rev Immunol 21, 233-243 (2021).
38. Tokunaga, A., et al. Selective inhibition of low-affinity memory CD8 (+) T cells by corticosteroids. J Exp Med 216, 2701-2713 (2019).
39. Wargnier, A., et al. Down-regulation of human granzyme B expression by glucocorticoids. Dexamethasone inhibits binding to the Ikaros and AP-1 regulatory elements of the granzyme B promoter. J Biol Chem 273, 35326-35331 (1998).
40. Groh, V., Wu, J., Yee, C. & Spies, T. Tumour-derived soluble MIC ligands impair expression of NKG2D and T-cell activation. Nature 419, 734-738 (2002).
41. Lopez-Soto, A., Gonzalez, S. & Galluzzi, L. Soluble NKG2D ligands limit the efficacy of immune checkpoint blockade. Oncoimmunology 6, e1346766 (2017).
42. Maccalli, C., et al. Soluble NKG2D ligands are biomarkers associated with the clinical outcome to immune checkpoint blockade therapy of metastatic melanoma patients. Oncoimmunology 6, e1323618 (2017).
43. Song, H., et al. IL-2/IL-18 prevent the down-modulation of NKG2D by TGF-beta in NK cells via the c-Jun N-terminal kinase (JNK) pathway. Cell Immunol 242, 39-45 (2006).
44. Faje, A. T., et al. High-dose glucocorticoids for the treatment of ipilimumab-induced hypophysitis is associated with reduced survival in patients with melanoma. Cancer 124, 3706-3714 (2018).
45. Long, M. D., et al. Risk of melanoma and nonmelanoma skin cancer among patients with inflammatory bowel disease. Gastroenterology 143, 390-399.e391 (2012).
46. Robinson, M. D., McCarthy, D. J. & Smyth, G. K. edgeR: a Bioconductor package for differential expression analysis of digital gene expression data. Bioinformatics 26, 139-140 (2009).
47. Hänzelmann, S., Castelo, R. & Guinney, J. GSVA: gene set variation analysis for microarray and RNA-Seq data. BMC Bioinformatics 14, 7 (2013).
48. Crowe, A. R. & Yue, W. Semi-quantitative Determination of Protein Expression using Immunohistochemistry Staining and Analysis: An Integrated Protocol. Bio Protoc 9, e3465 (2019).
49. Korotkevich, G., Sukhov, V. & Sergushichev, A. Fast gene set enrichment analysis. bioRxiv, 060012 (2019).

What is claimed is:

1. A method of treating an immunotherapy-associated adverse event in a subject in need thereof comprising
(a) detecting an elevated level of NKG2D receptor ligand polypeptide in a sample from the subject; and
(b) administering a corticosteroid to the subject.

2. The method of claim 1, wherein the NKG2D receptor ligand polypeptide is a soluble Major Histocompatibility Complex class I chain-related (sMIC) polypeptide or an UL binding protein (ULBP).

3. The method of claim 2, wherein the ULBP is ULBP-1, ULBP-2, UL-BP-3, ULBP-4, ULBP-5 or ULBP-6.

4. The method of claim 1, wherein the NKG2D receptor ligand polypeptide is a soluble Major Histocompatibility Complex class I chain-related (sMIC) polypeptide.

5. The method of claim 4, wherein the elevated level of sMIC in the sample comprises an amount that is ≥10% increase compared to baseline.

6. The method of any one of claims 1-5, wherein the immunotherapy-associated adverse event is colitis, skin toxicities including but not limiting to psoriatic, immunobullous, maculopapular, lichenoid, acantholytic eruptions, vitiligo, alopecias, vasculitides, and SJS/toxic epidermal necrolysis, neurotoxicity such as encephalitis, inflammatory arthritis, myocarditis, transverse myelitis, nephritis, myositis, Hepatotoxicity, Stevens-Johnson syndrome, Guillain-Barré syndrome, peripheral or autonomic neuropathy, Pneumonitis, Thrombocytopenia, or venous thromboembolism.

7. The method of any one of claims 1-6, wherein the subject has cancer.

8. The method of any one of claims 1-7, wherein the immunotherapy is an immune checkpoint inhibitor.

9. The method of claim 8, wherein the immune checkpoint inhibitor is a small molecule, an inhibitory nucleic acid, or an antibody.

10. The method of claim 8 or claim 9, wherein the immune checkpoint inhibitor is PD-L1, PD-L2, PD-1, CTLA-4, TIM-3, LAG-3, VISTA, or TIGIT.

11. The method of any one of claims 8-10, wherein the immune checkpoint inhibitor is PD-1.

12. The method of any one of claims 9-11, wherein the immune checkpoint inhibitor is an anti-PD-1 antibody.

13. The method of claim 12, wherein the anti-PD-1 antibody is pembrolizumab, nivolumab, or pidilizumab.

14. The method of any one of claims 9-10, wherein the immune checkpoint inhibitor is PD-L1.

15. The method of any one of claim 8-10 or 14, wherein the immune checkpoint inhibitor is an anti-PD-L1 antibody.

16. The method of claim 15, wherein the anti-PD-L1 antibody is atezolizumab, avelumab, or durvalumab.

17. The method of any one of claims 8-10, wherein the immune checkpoint is CTLA-4.

18. The method of any one of claim 8-9 or 17, wherein the immune checkpoint inhibitor is an anti-CTLA-4 antibody.

19. The method of claim 18, wherein the anti-CTLA-4 antibody is iplimumab.

20. The method of any one of claims 1-19, wherein the subject has received treatment with both an anti-PD-1 antibody and an anti-CTLA4 antibody.

21. The method of any one of claims 1-20, further comprising detecting an elevated level of IL-18 in the sample.

22. The method of claim 20, wherein the elevated level of IL-18 in the sample comprises an amount that is ≥90 pg/mL.

23. The method of any one of claims 1-21, wherein the sample is a serum sample, a tissue sample or a feces sample.

24. The method of any one of claims 1-23, wherein the tissue sample is a colon tissue sample.

25. The method of any one of claims 1-24, where step (a) comprises determining a level of sMIC protein in the sample and comparing the level of sMIC protein in the sample to a predetermined criterion.

26. The method of any one of claims 1-25, wherein the corticosteroid is prednisolone or prednisone.

27. A method of treating a immunotherapy-associated adverse event in a subject in need thereof comprising
(a) detecting a decreased level of NKG2D receptor ligand polypeptide in a sample from the subject, and
(b) administering a TNFα inhibitor or an integrin inhibitor to the subject.

28. The method of claim 27, wherein the NKG2D receptor ligand polypeptide is a soluble Major Histocompatibility Complex class I chain-related (sMIC) polypeptide or an UL binding protein (ULBP).

29. The method of claim 28, wherein the ULBP is ULBP-1, ULBP-2, UL-BP-3, ULBP-4, ULBP-5 or ULBP-6.

30. The method of claim 27, wherein the NKG2D receptor ligand polypeptide is a soluble Major Histocompatibility Complex class I chain-related (sMIC) polypeptide.

31. The method of claim 30, wherein the decreased level of sMIC in the sample comprises an amount that is ≥10% lower compared to baseline.

32. The method of any one of claims 27-31, wherein the immunotherapy-associated adverse event is colitis, skin toxicities including but not limiting to psoriatic, immunobullous, maculopapular, lichenoid, acantholytic eruptions, vitiligo, alopecias, vasculitides, and SJS/toxic epidermal necrolysis, neurotoxicity such as encephalitis, inflammatory arthritis, myocarditis, transverse myelitis, nephritis, myositis, Hepatotoxicity, Stevens-Johnson syndrome, Guillain-Barré syndrome, peripheral or autonomic neuropathy, Pneumonitis, Thrombocytopenia, or Venous thromboembolism.

33. The method of any one of claims 27-32, wherein the subject has cancer.

34. The method of any one of claims 27-33, wherein the immunotherapy is an immune checkpoint inhibitor.

35. The method of claim 34, wherein the immune checkpoint inhibitor is a small molecule, an inhibitory nucleic acid, or an antibody.

36. The method of claim 34 or claim 35, wherein the immune checkpoint is PD-L1, PD-L2, PD-1, CTLA-4, TIM-3, LAG-3, VISTA, or TIGIT.

37. The method of any one of claims 34-36, wherein the immune checkpoint is PD-1.

38. The method of any one of claims 34-37, wherein the immune checkpoint inhibitor is an anti-PD-1 antibody.

39. The method of claim 38, wherein the anti-PD-1 antibody is pembrolizumab, nivolumab, or pidilizumab.

40. The method of any one of claims 34-36, wherein the immune checkpoint inhibitor is PD-L1.

41. The method of any one of claim 34-36 or 40, wherein the immune checkpoint inhibitor is an anti-PD-L1 antibody.

42. The method of claim 41, wherein the anti-PD-L1 antibody is atezolizumab, avelumab, or durvalumab.

43. The method of any one of claims 34-36, wherein the immune checkpoint inhibitor is CTLA-4.

44. The method of any one of claim 34-36 or 43, wherein the immune checkpoint inhibitor is an anti-CTLA-4 antibody.

45. The method of claim 44, wherein the anti-CTLA-4 antibody is iplimumab.

46. The method of any one of claims 27-45, wherein the subject has received treatment with both an anti-PD-1 antibody and an anti-CTLA4 antibody.

47. The method of any one of claims 34-46, wherein step (a) comprises determining a level of sMIC protein in the sample and comparing the level of sMIC protein in the sample to a predetermined criterion.

48. The method of any one of claims 34-47, wherein the TNFα inhibitor is infliximab or adalimumab.

49. The method of any one of claims 27-48, wherein the integrin inhibitor is vedolizumab.


SEQUENCE LISTING (90 sequences)

SEQ ID NO: 1 - protein, 383 aa, Homo sapiens
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 2 - protein, 383 aa, Homo sapiens
MGLGRVLLFLAVAFPFAPPAAAAEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGT

SEQ ID NO: 3 - protein, 274 aa, Homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEALEGNITVTCGASSFYPRNITLTWRQDGVSLSHDTQQWGDVLPDGNGTYQTWVATRICQGEEQRFTCYMEHSGNHSTHPVPS

SEQ ID NO: 4 - protein, 80 aa, Homo sapiens
TCRASGFYPWNITLSWRQDGVSLSHDTQQWGDVLPDGNGTYQTWVATRICQGEEQRFTCYMEHSGNHSTHPVPSGKVLVL

SEQ ID NO: 5 - protein, 80 aa, Homo sapiens; identical to SEQ ID NO: 4

SEQ ID NO: 6 - protein, 74 aa, Homo sapiens
TCRASGFYPWNITLSWRQDGVSLSHDTQQWGDVLPDGNGTYQTWVATRICQGEEQRFTCYMEHSGNHSTHPVPS

SEQ ID NO: 7 - protein, 80 aa, Homo sapiens
TCRASSFYPRNITLTWRQDGVSLSHDTQQWGDVLPDGNGTYQTWVATRICQGEEQRFTCYMEHSGNHSTHPVPSGKVLVL

SEQ ID NO: 8 - protein, 74 aa, Homo sapiens
TCRASSFYPRNITLTWRQDGVSLSHDTQQWGDVLPDGNGTYQTWVATRICRGEEQRFTCYMEHSGNHSTHPVPS

SEQ ID NO: 9 - protein, 80 aa, Homo sapiens; identical to SEQ ID NO: 7

SEQ ID NO: 10 - protein, 80 aa, Homo sapiens; identical to SEQ ID NO: 4

SEQ ID NO: 11 - protein, 74 aa, Homo sapiens; identical to SEQ ID NO: 6

SEQ ID NO: 12 - protein, 80 aa, Homo sapiens; identical to SEQ ID NO: 4

SEQ ID NO: 13 - protein, 81 aa, Homo sapiens
TCRASSFYPRNIITTSWRQDGVSLSHDTQQWGDVLPDGNGTYQTWVATRICRGEEQRFTCYMEHSGNHSTHPVPSGKVLVL

SEQ ID NO: 14 - protein, 81 aa, Homo sapiens; identical to SEQ ID NO: 13

SEQ ID NO: 15 - protein, 75 aa, Homo sapiens
TCRASSFYPRNIITTSWRQDGVSLSHDTQQWGDVLPDGNGTYQTWVATRICRGEEQRFTCYMEHSGNHSTHPVPS

SEQ ID NO: 16 - protein, 80 aa, Homo sapiens; identical to SEQ ID NO: 7

SEQ ID NO: 17 - protein, 80 aa, Homo sapiens; identical to SEQ ID NO: 7

SEQ ID NO: 18 - protein, 80 aa, Homo sapiens
TCRASSFYPRNIILTWRQDGVSLSHDTQQWGDVLPDGNGTYQTWVATRICRGEEQRFTCYMEHSGNHSTHPVPSGKVLVL

SEQ ID NO: 19 - protein, 80 aa, Homo sapiens
TCRASGFYPWNITLSWRQDGVSLSHDTQQWGDVLPDGNGTYQTWVATRICQGEEQRFTCYMEHSGNHSTHAVPSGKVLVL

SEQ ID NO: 20 - protein, 80 aa, Homo sapiens; identical to SEQ ID NO: 4

SEQ ID NO: 21 - protein, 74 aa, Homo sapiens; identical to SEQ ID NO: 6

SEQ ID NO: 22 - protein, 74 aa, Homo sapiens
TCRASGFYPWNITLSWRQDGVSLSHDTQQWGDVLPDGNGTYQTWVATRICRGEEQRFTCYMEHSGNHSTHPVPS

SEQ ID NO: 23 - protein, 74 aa, Homo sapiens; identical to SEQ ID NO: 6

SEQ ID NO: 24 - protein, 80 aa, Homo sapiens
TCRASGFYPWNITLSWRQDGVSLSHDTQQWGDVLPDGNGTYQTWVATRICQGEEQRFTCYMEHSGNHSTHPVPSGKCWCF

SEQ ID NO: 25 - protein, 80 aa, Homo sapiens
TCRASSFYPRNIILTWRQDGLSLSHDTQQWGDVLPDGNGTYQTWVATRICRGEEQRFTCYMEHSGNHSTHPVPSGKVLVL

SEQ ID NO: 26 - protein, 80 aa, Homo sapiens; identical to SEQ ID NO: 24

SEQ ID NO: 27 - protein, 80 aa, Homo sapiens; identical to SEQ ID NO: 4

SEQ ID NO: 28 - protein, 80 aa, Homo sapiens; identical to SEQ ID NO: 4

SEQ ID NO: 29 - protein, 81 aa, Homo sapiens
TCRASSFYPRNIILTSWRQDGVSLSHDTQQWGDVLPDGNGTYQTWVATRICRGEEQRFTCYMEHSGNHSTHPVPSGKVLVL

SEQ ID NO: 30 - protein, 80 aa, Homo sapiens; identical to SEQ ID NO: 4

SEQ ID NO: 31 - protein, 74 aa, Homo sapiens; identical to SEQ ID NO: 6

SEQ ID NO: 32 - protein, 74 aa, Homo sapiens
TCRASSFYPRNIILTWRQDGVSLSHDTQQWGDVLPDGNGTYQTWVATRICRGEEQRFTCYMEHSGNHSTHPVPS

SEQ ID NO: 33 - protein, 80 aa, Homo sapiens; identical to SEQ ID NO: 4

SEQ ID NO: 34 - protein, 74 aa, Homo sapiens
TCRASSFYPRNITLTWRQDGVSLSHDTQQWGDVLPDGNGTYQTWVATRICQGEEQRFTCYMEHSGNHSTHPVPS

SEQ ID NO: 35 - protein, 74 aa, Homo sapiens; identical to SEQ ID NO: 6

SEQ ID NO: 36 - protein, 80 aa, Homo sapiens; identical to SEQ ID NO: 4

SEQ ID NO: 37 - protein, 80 aa, Homo sapiens; identical to SEQ ID NO: 18

SEQ ID NO: 38 - protein, 80 aa, Homo sapiens; identical to SEQ ID NO: 4

SEQ ID NO: 39 - protein, 80 aa, Homo sapiens; identical to SEQ ID NO: 4

SEQ ID NO: 40 - protein, 74 aa, Homo sapiens
TCRASGFYPWNITLSWRQDGVSLSHDTQQWGDVLPDGNGTYQTWVATRICQGEEQRFTCYMEHSGNHSTHAVPS

SEQ ID NO: 41 - protein, 74 aa, Homo sapiens; identical to SEQ ID NO: 6

SEQ ID NO: 42 - protein, 74 aa, Homo sapiens; identical to SEQ ID NO: 6

SEQ ID NO: 43 - protein, 81 aa, Homo sapiens
TCRASSFYPRNITLTSWRQDGVSLSHDTQQWGDVLPDGNGTYQTWVATRICRGEEQRFTCYMEHSGNHSTHPVPSGKVLVL

SEQ ID NO: 44 - protein, 75 aa, Homo sapiens
TCRASSFYPRNITLTSWRQDGVSLSHDTQQWGDVLPDGNGTYQTWVATRICQGEEQRFTCYMEHSGNHSTHPVPS

SEQ ID NO: 45 - protein, 74 aa, Homo sapiens; identical to SEQ ID NO: 32

SEQ ID NO: 46 - protein, 74 aa, Homo sapiens; identical to SEQ ID NO: 6

SEQ ID NO: 47 - protein, 74 aa, Homo sapiens; identical to SEQ ID NO: 32

SEQ ID NO: 48 - protein, 74 aa, Homo sapiens; identical to SEQ ID NO: 34

SEQ ID NO: 49 - protein, 75 aa, Homo sapiens
TCRASSFYPRNIILTWRQDGLSLSHDTQQWGDVLPDGNGTYQTWVATRICRQGEEQRFTCYMEHSGNHSTHPVPS

SEQ ID NO: 50 - protein, 74 aa, Homo sapiens; identical to SEQ ID NO: 6

SEQ ID NO: 51 - protein, 80 aa, Homo sapiens; identical to SEQ ID NO: 4

SEQ ID NO: 52 - protein, 74 aa, Homo sapiens
TCRASRFYPRNIILTWRQDGVSLSHDTQQWGDVLPDGNGTYQTWVATRICRGEEQRFTCYMEHSGNHSTHPVPS

SEQ ID NO: 53 - protein, 80 aa, Homo sapiens
TCRASGFYPWNITLSWRQDGVSLSHDTQQWGDVLPDGNGTYQTWVATRICQGEEQSFTCYMEHSGNHSTHPVPSGKVLVL

SEQ ID NO: 54 - protein, 74 aa, Homo sapiens; identical to SEQ ID NO: 34

SEQ ID NO: 55 - protein, 80 aa, Homo sapiens
TCRASGFYPWNITLSWRQDGVSLSHDTQQWGDVLPDGNGTYQTWVATRICEGEEQRFTCYMEHSGNHSTHPVPSGKVLVL

SEQ ID NO: 56 - protein, 80 aa, Homo sapiens
TCRASGFCPWNITLSWRQDGVSLSHDTQQWGDVLPDGNGTYQTWVATRICQGEEQRFTCYMEHSGNHSTHPVPSGKVLVL

SEQ ID NO: 57 - protein, 80 aa, Homo sapiens; identical to SEQ ID NO: 19

SEQ ID NO: 58 - protein, 80 aa, Homo sapiens; identical to SEQ ID NO: 18

SEQ ID NO: 59 - protein, 81 aa, Homo sapiens
TCRASSFYPRNITLTSWRQDGVSLSHDTQQWGDVLPDGNGTYQTWVATRICQGEEQRFTCYMEHSGNHSTHPVPSGKVLVL

SEQ ID NO: 60 - protein, 80 aa, Homo sapiens; identical to SEQ ID NO: 4

SEQ ID NO: 61 - protein, 80 aa, Homo sapiens; identical to SEQ ID NO: 4

SEQ ID NO: 62 - protein, 80 aa, Homo sapiens
TCRASSFYPRNITLTWRQDGVSLSHNTQQWGDVLPDGNGTYQTWVATRIRQGEEQRFTCYMEHSGNHGTHPVPSGKVLVL

SEQ ID NO: 63 - protein, 80 aa, Homo sapiens
TCRASSFYPRNITLTWRQDGVSLSHNTQQWGDVLPDGNGTYQTWVATRIRQGEEQRFTCYMEHSGNHGTHPVPSGKALVL

SEQ ID NO: 64 - protein, 80 aa, Homo sapiens; identical to SEQ ID NO: 63

SEQ ID NO: 65 - protein, 80 aa, Homo sapiens; identical to SEQ ID NO: 63

SEQ ID NO: 66 - protein, 80 aa, Homo sapiens; identical to SEQ ID NO: 63

SEQ ID NO: 67 - protein, 80 aa, Homo sapiens; identical to SEQ ID NO: 63

SEQ ID NO: 68 - protein, 80 aa, Homo sapiens; identical to SEQ ID NO: 63

SEQ ID NO: 69 - protein, 80 aa, Homo sapiens; identical to SEQ ID NO: 63

SEQ ID NO: 70 - protein, 80 aa, Homo sapiens; identical to SEQ ID NO: 63

SEQ ID NO: 71 - protein, 80 aa, Homo sapiens
TCRASSFYPRNITLTWRQDGVSLSHNTQQWGDVLPDGNGTYQTWVATRIRQGEEQKFTCYMEHSGNHGTHPVPSGKALVL

SEQ ID NO: 72 - protein, 80 aa, Homo sapiens; identical to SEQ ID NO: 63

SEQ ID NO: 73 - protein, 80 aa, Homo sapiens; identical to SEQ ID NO: 63

SEQ ID NO: 74 - protein, 80 aa, Homo sapiens; identical to SEQ ID NO: 63

SEQ ID NO: 75 - protein, 80 aa, Homo sapiens; identical to SEQ ID NO: 63

SEQ ID NO: 76 - protein, 80 aa, Homo sapiens
TCRASSFYPRNITLTWRQDGVSLSHNTQQWGDVLPDGNGTYQTWVATRIRQGEEQRFTCYMEHSGNHSTHPVPSGKALVL

SEQ ID NO: 77 - protein, 80 aa, Homo sapiens; identical to SEQ ID NO: 76

SEQ ID NO: 78 - protein, 80 aa, Homo sapiens; identical to SEQ ID NO: 76

SEQ ID NO: 79 - protein, 74 aa, Homo sapiens
TCRASSFYPRNITLTWRQDGVSLSHNTQQWGDVLPDGNGTYQTWVATRIRQGEEQRFTCYMEHSGNHSTHPVPS

SEQ ID NO: 80 - protein, 74 aa, Homo sapiens
TCRASSFYPRNITLTWRQDGVSLSHNTQQWGDVLPDGNGTYQTWVATRIRQGEEQRFTCYMEHSGNHGTHPVPS

SEQ ID NO: 81 - protein, 74 aa, Homo sapiens; identical to SEQ ID NO: 80

SEQ ID NO: 82 - protein, 74 aa, Homo sapiens; identical to SEQ ID NO: 80

SEQ ID NO: 83 - protein, 74 aa, Homo sapiens; identical to SEQ ID NO: 80

SEQ ID NO: 84 - protein, 74 aa, Homo sapiens; identical to SEQ ID NO: 80

SEQ ID NO: 85 - protein, 244 aa, Homo sapiens
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 86 - protein, 246 aa, Homo sapiens
MAAAAATKILLCLPLLLLLSGWSRAGRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMSSGTTQLRATATTLILCCLLIILPCFILPGI

SEQ ID NO: 87 - protein, 244 aa, Homo sapiens
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 88 - protein, 263 aa, Homo sapiens
MRRISLTSSPVRLLLFLLLLLIALEIMVGGHSLCFNFTIKSLSRPGQPWCEAQVFLNKNLFLQYNSDNNMVKPLGLLGKKVYATSTWGELTQTLGEVGRDLRMLLCDIKPQIKTSDPSTLQVEMFCQREAERCTGASWQFATNGEKSLLFDAMNMTWTVINHEASKIKETWKKDRGLEKYFRKLSKGDCDHWLREFLGHWEAMPEPTVSPVNASDIHWSSSSLPDRWIILGAFILLVLMGIVLICVWWQNGEWQAGLWPLRTS

SEQ ID NO: 89 - protein, 334 aa, Homo sapiens
MAAAASPAFLLRLPLLLLLSSWCRTGLADPHSLCYDITVIPKFRPGPRWCAVQGQVDEKTFLHYDCGSKTVTPVSPLGKKLNVTTAWKAQNPVLREVVDILTEQLLDIQLENYIPKEPLTLQARMSCEQKAEGHGSGSWQLSFDGQIFLLFDSENRMWTTVHPGARKMKEKWENDKDMTMSFHYISMGDCTGWLEDFLMGMDSTLEPSAGAPPTMSSGTAQPRATATTLILCCLLIMCLLICSRHSLTQSHGHHPQSLQPPPHPPLLHPTWLLRRVLWSDSYQIAKRPLSGGHVTRVTLPIIGDDSHSLPCPLALYTINNGAARYSEPLQVSIS

SEQ ID NO: 90 - protein, 246 aa, Homo sapiens
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI